(19) United States
(12) Patent Application Publication
(10) Pub. No.: US 2025/0261988 A1
(43) Pub. Date: Aug. 21, 2025

(54) Electrosurgical Instrument

(71) Applicant: Stryker European Operations Limited, Cork (IE)

(72) Inventors: Micheal Burke, Cork (IE); Paul Sheridan, Wexford (IE); Laura Constance Frey, Belfast (IE); Scott McFarland, Greenisland (IE)

(21) Appl. No.: 18/854,872

(22) PCT Filed: Apr. 11, 2023
(86) PCT No.: PCT/IB2023/000195

§ 371 Date: Oct. 7, 2024

Related U.S. Application Data
(60) Provisional application No. 63/329,738, filed on Apr. 11, 2022.
(60) Provisional application No. 63/428,310, filed on Nov. 28, 2022.

(51) Int. Cl. A61B 18/14; A61B 17/00; A61B 18/00

(52) U.S. Cl. CPC A61B 18/148; A61B 2017/0042; A61B 2017/00526; A61B 2018/00607; A61B 2018/00708; A61B 2018/00922; A61B 2018/00946

(57) ABSTRACT

An example electrosurgical instrument includes a housing extending from a proximal end to a distal end, an electrosurgical electrode extending from the distal end of the housing, and a user activation device disposed between the proximal end of the housing and the distal end of the housing. The user activation device is operable to control a supply of electrosurgical energy to the electrosurgical electrode. The user activation device includes a collar that extends around at least a half of a circumference of the housing. The collar is movable between a home position relative to the housing and a first activation position relative to the housing. The user activation device also includes a switch that is configured to control the supply of electrosurgical energy to the electrosurgical electrode responsive to the collar moving from the home position to the first activation position.

### CROSS-REFERENCE TO RELATED APPLICATIONS

This application claims the benefit of priority to U.S. Provisional Application No. 63/329,738, filed Apr. 11, 2022, and U.S. Provisional Application No. 63/428,310, filed Nov. 28, 2022, the contents of which applications are hereby incorporated by reference in their entirety.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”). As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative implementation of the present disclosure when read in conjunction with the accompanying figures, wherein:

FIG. 1 depicts a simplified block diagram of an electrosurgical system, according to an example.

FIG. 2A depicts an electrosurgical instrument of FIG. 1 with a collar in a home position, according to an example.

FIG. 2B depicts the electrosurgical instrument of FIG. 2A with the collar in a first activation position, according to an example.

FIG. 2C depicts the electrosurgical instrument of FIG. 2A with the collar in the second activation position, according to the example.

FIG. 3A depicts an electrosurgical instrument of FIG. 1 with a collar in a home position, according to another example.

FIG. 3B depicts the electrosurgical instrument of FIG. 3A with the collar in a first activation position, according to an example.

FIG. 3C depicts the electrosurgical instrument of FIG. 3A with the collar in the second activation position, according to the example.

FIG. 4A depicts an electrosurgical instrument of FIG. 1 with a collar in a home position, according to another example.

FIG. 4B depicts the electrosurgical instrument of FIG. 4A with the collar in a first activation position, according to an example.

FIG. 4C depicts the electrosurgical instrument of FIG. 4A with the collar in the second activation position, according to the example.

FIG. 5A depicts an electrosurgical instrument of FIG. 1 with a collar in a home position, according to another example.

FIG. 5B depicts the electrosurgical instrument of FIG. 5A with the collar in a first activation position, according to an example.

FIG. 5C depicts the electrosurgical instrument of FIG. 5A with the collar in the second activation position, according to the example.

FIG. 6A depicts a perspective view of a first example implementation of the electrosurgical instrument shown in FIG. 1, according to another example.

FIG. 6B depicts a side view of the electrosurgical instrument shown in FIG. 6A, according to an example.

FIG. 6C depicts a perspective view of a second example implementation of the electrosurgical instrument shown in FIG. 1, according to another example.

FIG. 6D depicts a side view of the electrosurgical instrument shown in FIG. 6C, according to an example.

FIG. 6E depicts a distal end of a shaft of the electrosurgical instrument of FIGS. 6A-6D with a suction sleeve removed, according to an example.

FIG. 7A depicts a perspective view of a user activation device including collar having a plurality of independently movable segments, according to an example.

FIG. 7B depicts a side view of the collar and a plurality of switches for the user activation device shown in FIG. 7A, according to an example.

FIG. 7C depicts the collar of FIG. 7A in a first activation position, according to an example.

FIG. 7D depicts the collar of FIG. 7A in a second activation position, according to the example.

FIG. 8 depicts a flowchart for a method for operating an electrosurgical instrument, according to an example.

FIG. 9 depicts a flowchart for a method of operating an electrosurgical instrument that can be used with the method shown in at least FIG. 8, according to an example.

FIG. 10 depicts a flowchart for a method of operating an electrosurgical instrument that can be used with the method shown in at least FIG. 8, according to an example.

FIG. 11 depicts a flowchart for a method of operating an electrosurgical instrument that can be used with the method shown in at least FIG. 10, according to an example.

FIG. 12 depicts a flowchart for a method of operating an electrosurgical instrument that can be used with the method shown in at least FIG. 11, according to an example.

FIG. 13 depicts a flowchart for a method of operating an electrosurgical instrument that can be used with the method shown in at least FIG. 8, according to an example.

FIG. 14 depicts a flowchart for a method of operating an electrosurgical instrument that can be used with the method shown in at least FIG. 8, according to an example.

FIG. 15 depicts a flowchart for a method of operating an electrosurgical instrument that can be used with the method shown in at least FIG. 8, according to an example.

FIG. 16 depicts a flowchart for a method of operating an electrosurgical instrument that can be used with the method shown in at least FIG. 8, according to an example.

FIG. 17 depicts a flowchart for a method for operating an electrosurgical instrument, according to an example.

FIG. 18 depicts a simplified block diagram of an electrosurgical system, according to an example.

FIG. 19A depicts a side view of an electrosurgical device, according to an example.

FIG. 19B depicts a perspective view of the electrosurgical device of FIG. 19A, according to an example.

FIG. 19C depicts a cross-sectional view of the electrosurgical device of FIG. 19A, according to an example.

FIG. 20 illustrates an example implementation of an electrosurgical device, according to an example.

FIG. 21 depicts a flowchart for a method for operating an electrosurgical device, according to an example.

FIG. 22 depicts a simplified block diagram of a surgical instrument, according to an example.

### DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

During an electrosurgical operation, it may be beneficial to adjust a rotational position of an electrosurgical electrode relative to a position of a user activation device on a handle of an electrosurgical instrument. This can allow a user to comfortably grip the handle in a position in which their fingers can comfortably operate the user activation devices while the electrosurgical electrode is set at a rotational position selected from among a plurality of rotational positions relative to the handle based on, for example, a location, a size, and/or a shape of a surgical site in which the user is operating. Conventionally, this is accomplished by stopping an operation, manually rotating the electrosurgical electrode relative to the handle using both of the user's hands (e.g., one hand to hold the handle and the other hand to rotate the electrosurgical electrode), and then resuming the operation.

The present disclosure provides for electrosurgical instruments that can address at least some of the challenges associated with electrosurgical instruments that require manual adjustment of the electrosurgical electrode relative to the handle. As disclosed herein, electrosurgical instruments, such as electrosurgical pencils, allow a user to operate a user activation device from multiple angles or approaches.

In some examples, the user activation device can include a collar that extends around at least a half of a circumference of a housing the electrosurgical instrument and is movable relative to the housing between a home position on the housing and at least a first activation position on the housing. The user activation device also includes a switch, and the collar can move relative to the switch (e.g., collar can slide over the switch). The switch is configured to prevent a supply of electrosurgical energy to an electrosurgical electrode when the collar is in the home position, and cause the supply of the electrosurgical energy to the electrosurgical electrode when the collar is in the first activation position. Because the collar extends around at least half of the circumference of the housing, the switch can be operated in a plurality of rotational orientations of the housing relative to the hand of the user and regardless of a position of the hand of the user relative to the switch underlying the collar. This provides the user with the capability of holding an electrosurgical instrument in a manner that yields a preferred position and orientation for performing an electrosurgical operation (e.g., cutting, coagulation, etc.) at a surgical site, while comfortably operating the user activation device according to the mode of operation. Advantageously, this reduces the need to stop and restart a surgical procedure to allow the user to adjust the electrosurgical electrode to maintain the preferred position and orientation for the electrosurgical electrode. Furthermore, this user activation device provides ambidextrous functionality (i.e., accommodates operation by right or left hand).

In other examples, electrosurgical devices, such as electrosurgical pencils, allow a user to depress and operate a user activation device from multiple angles or approaches. For example, a multi-axial user activation device provides the user with the capability of holding an electrosurgical device in a manner that yields a preferred position and orientation for performing an electrosurgical operation (e.g., cutting, coagulation, etc.) at a surgical site, while comfortably operating the multi-axial user activation device according to the mode of operation. Advantageously, this reduces the need to stop and restart a surgical procedure to allow the user to adjust the electrosurgical electrode to maintain the preferred position and orientation for the electrosurgical electrode. Furthermore, this multi-axial user activation device provides ambidextrous functionality (i.e., accommodates operation by right or left hand).

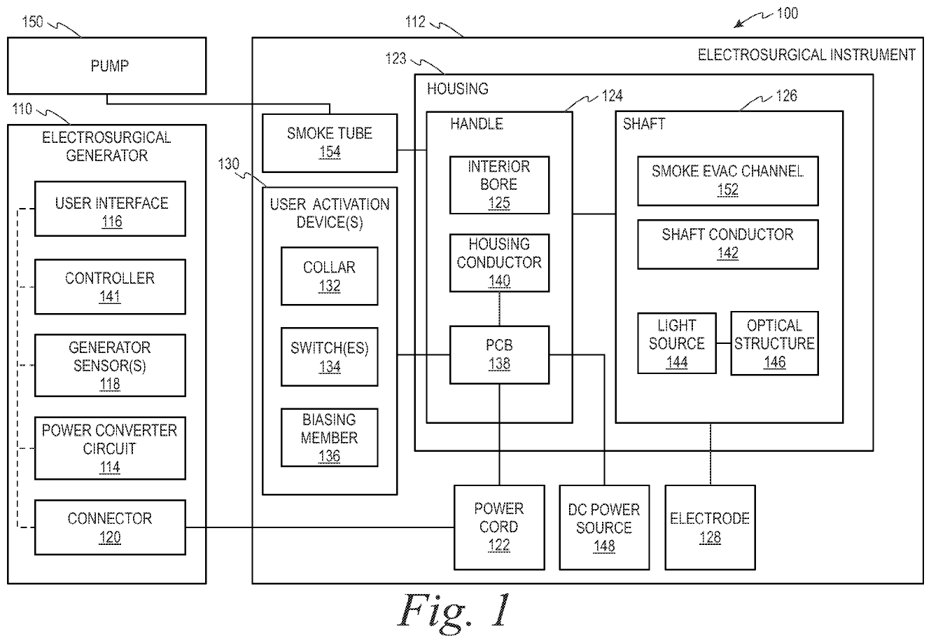

Referring to FIG. 1, an electrosurgical system 100 is shown according to an example. As shown in FIG. 1, the electrosurgical system 100 includes an electrosurgical generator 110 and an electrosurgical instrument 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, one or more display screens, one or more indicator lights, one or more speakers, and/or one or more haptic output devices.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more sensors 118 that can sense one or more conditions related to the electrosurgical energy and/or the target tissue. As examples, the sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 kHz and approximately 500 kHz.

In FIG. 1, the electrosurgical generator 110 also includes a connector 120 that can facilitate coupling the electrosurgical generator 110 to the electrosurgical instrument 112. For example, the electrosurgical instrument 112 can include a power cord 122 having a plug, which can be coupled to a socket of the connector 120 of the electrosurgical generator 110. In this arrangement, the electrosurgical generator 110 can supply the electrosurgical energy to the electrosurgical instrument 112 via the coupling between the connector 120 of the electrosurgical generator 110 and the power cord 122 of the electrosurgical instrument 112.

The electrosurgical generator 110 can further include a controller 141 that can control operation of the electrosurgical generator 110. Within examples, the controller 141 can be implemented using hardware, software, and/or firmware. For instance, the controller 141 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 141, thus, can receive data and store the data in the memory as well. As shown in FIG. 1, the controller 141 can be communicatively coupled with the power converter circuit 114, the user interface 116, the generator sensor(s) 118, and/or the connector 120.

As shown in FIG. 1, the electrosurgical instrument 112 can include a housing 123. The housing 123 can be an elongated structure in and/or on which components of the electrosurgical instrument 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior bore, a shaft 126 extending in a distal direction from the handle 124, and an electrosurgical electrode 128 coupled to the shaft 126. In general, the handle 124 can be configured to facilitate a user gripping and manipulating the electrosurgical instrument 112 while performing electrosurgery. For example, the handle 124 can have a shape and/or a size that can facilitate a user performing electrosurgery by manipulating the electrosurgical instrument 112 using a single hand. In one implementation, the handle 124 can have a shape and/or a size that facilitates the user holding the electrosurgical instrument 112 in a writing utensil gripping manner (e.g., the electrosurgical instrument 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical instrument 112 while performing the electrosurgery.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In one example, the handle 124 and the shaft 126 can be formed as a single, monolithic structure such that the shaft 126 and the handle 124 are fixed and non-moveable relative to each other. In another example, the handle 124 and the shaft 126 can be fixedly coupled to each other by a welding coupling, an adhesive coupling, and/or another coupling that prevents movement between the handle 124 and the shaft 126.

In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical instrument 112). In some examples, the electrosurgical electrode 128 can be coupled to the shaft 126 and, thus, the electrosurgical electrode 128 can move together with the shaft 126 in an axial direction along the longitudinal axis relative to the handle 124. This can provide for adjusting a length of the electrosurgical instrument 112, which can facilitate performing electrosurgery at a plurality of different depths within tissue (e.g., due to different anatomical shapes and/or sizes of patients) and/or at a plurality of different angles. In other examples, the electrosurgical electrode 128 can be fixedly coupled to the handle 124 such that the shaft 126 is axially movable relative to both the electrosurgical electrode 128 and the handle 124.

The electrosurgical electrode 128 can be rotationally fixed relative to the handle 124 and/or the shaft 126. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). As described in further detail below, the user activation device(s) of the electrosurgical instrument 112 described herein can help to reduce or obviate a need to rotate the electrosurgical electrode 128 relative to the handle 124 and/or the shaft 126.

As shown in FIG. 1, the electrosurgical instrument 112 can include one or more user activation devices 130 that are operable to control operation of the electrosurgical instrument 112 and/or the electrosurgical generator 110. For instance, the user activation device(s) 130 can be operable to select between the modes of operation of the electrosurgical instrument 112 and/or the electrosurgical generator 110. In one implementation, the user activation device(s) 130 can be configured to select between a cutting mode of operation and a coagulation mode of operation. Responsive to actuation of the user activation device(s) 130 of the electrosurgical instrument 112, the electrosurgical instrument 112 can (i) receive the electrosurgical energy with a level of power and/or a waveform corresponding to the mode of operation selected via the user activation device(s) 130 and (ii) supply the electrosurgical energy to the electrosurgical electrode 128.

The user activation device(s) 130 can be disposed between a proximal end of the housing 123 and a distal end of the housing 123. As shown in FIG. 1, the user activation device(s) 130 includes a collar 132 and one or more switches 134. Within examples, the collar 132 extends around at least a half of a circumference of the housing 123. This can allow a user to easily and quickly move a hand to a different rotational position on the housing 123 (e.g., on the handle 124) to adjust a rotational orientation of the hand relative to the electrosurgical electrode 128 while allowing the user to continue to using a preferred grip for performing an electrosurgical operation (e.g., cutting, coagulation, etc.) at a surgical site. Advantageously, this reduces the need to stop and restart a surgical procedure to allow the user to adjust the electrosurgical electrode 128 to maintain the preferred position and orientation for the electrosurgical electrode 128 as required by the conventional electrosurgical instruments described above. Additionally or alternatively, the collar 132 extending around at least half of the circumference of the housing 123 can provide for ambidextrous functionality (i.e., accommodates operation by right or left hand).

In some examples, the collar 132 extends around an entirety of the circumference of the housing 123. This can allow the user to hold the housing 123 in all rotational orientations relative to the hand (e.g., 360 degrees of rotational positions) while allowing the user to continue to using a preferred grip for performing an electrosurgical operation (e.g., cutting, coagulation, etc.) at a surgical site.

The collar 132 is movable between a home position relative to the housing 123 and a first activation position relative to the housing 123. The switch(es) 134 are configured to control the supply of the electrosurgical energy to the electrosurgical electrode 128 responsive to the collar 132 moving from the home position to the first activation position. For example, responsive to the collar 132 moving from the home position to the first activation position, the switch(es) 134 can cause the electrosurgical energy to be supplied to the electrosurgical electrode 128 with a first waveform (e.g., for cutting tissue or for coagulating tissue). By contrast, when the collar 132 is in the home position, the switch(es) 134 can prevent the electrosurgical energy from being supplied to the electrosurgical electrode 128.

In some examples, the user activation device 130 can also include a biasing member 136 that can bias the collar 132 towards the home position. The biasing member 136 can thus help to mitigate inadvertent supply of the electrosurgical energy to the electrosurgical electrode 128. As examples, the biasing member 136 can include one or more components selected from among a group consisting of: a spring, a magnet, and a mechanical engagement between respective surfaces of the collar 132 and the housing 123 (e.g., ramped surfaces biasing the collar 132 toward the home position).

In some examples, the collar 132 can be further movable between the home position and a second activation position relative to the housing 123. In such examples, the home position is between the first activation position and the second activation position. Responsive to the collar 132 moving from the home position to the second activation position, the switch(es) 134 can cause the electrosurgical energy to be supplied to the electrosurgical electrode 128 with a second waveform. The first waveform is different from the second waveform. For instance, in one example, the first waveform can be suitable for a cutting operation and the second waveform can be suitable for a coagulation operation. In another example, the first waveform can be suitable for a coagulation operation and the second waveform can be suitable for a cutting operation

In examples, in which the collar 132 is movable between the home position and the second activation position and the user activation device 130 includes the biasing member 136, the biasing member 136 can be further configured to bias the collar 132 toward the home position from the first activation position and the second activation position. As described above, when the collar 132 is in the home position, the switch(es) 134 can prevent the electrosurgical energy from being supplied to the electrosurgical electrode 128.

Within examples, the first activation position can be distal of the home position and the second activation position is proximal of the home position. Additionally, the collar 132 can be movable by sliding the collar 132 along an axis that is parallel or collinear with a longitudinal axis of the handle 124 (e.g., an axis extending between the proximal end and the distal end of the handle 124).

In FIG. 1, the electrosurgical instrument 112 includes a plurality of electrical components that facilitate supplying the electrosurgical energy, which the electrosurgical instrument 112 receives from the electrosurgical generator 110, to the electrosurgical electrode 128. For example, the electrosurgical instrument 112 can include at least one electrical component selected from a group of electrical components including: the switch(es) 134, a printed circuit board 138 (e.g., a flexible printed circuit board), a housing conductor 140, and/or a shaft conductor 142 that can provide a circuit for conducting the electrosurgical energy from the power cord 122 to the electrosurgical electrode 128. One or more of the electrical components can be positioned in the inner bore defined by the handle 124 and/or in an inner cavity defined by the shaft 126.

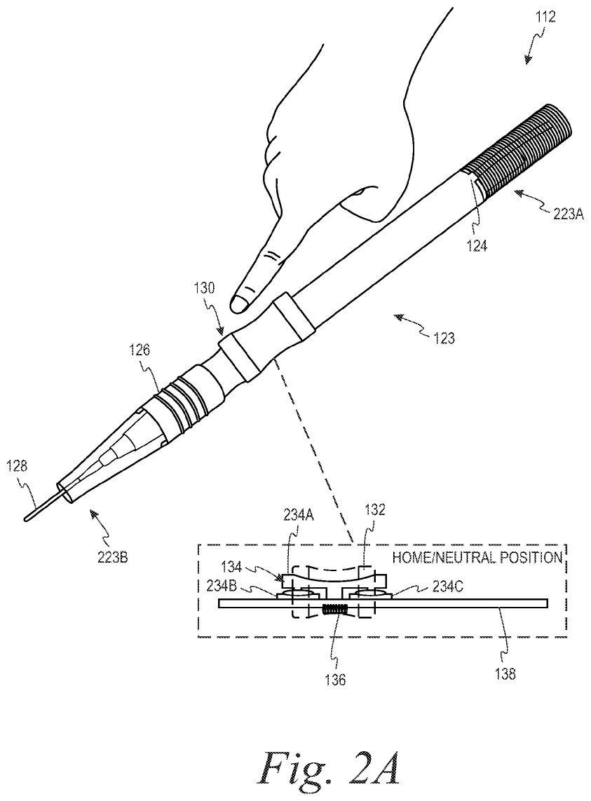

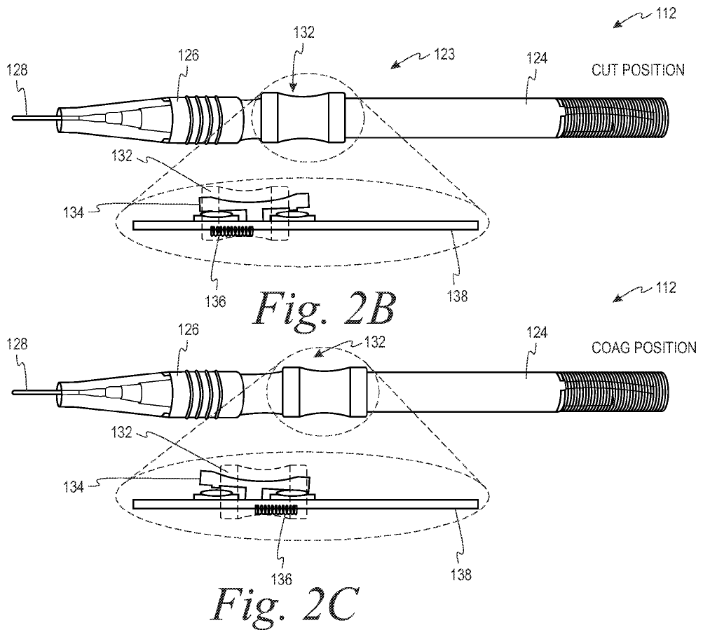

As described in further detail below with respect to FIGS. 2A-7D, the collar 132 is operable to actuate the switch(es) 134. As examples, the switch(es) 134 can include one or more components selected from a group consisting of: a rocker switch, a sensor (e.g., a hall effect sensor), a plurality of electrical contacts, and a plurality of piezoelectric devices.

The switch(es) 134 alone or in combination with the printed circuit board 138 can be operable to control a supply of the electrosurgical energy from the electrosurgical generator 110 to the electrosurgical electrode 128. For instance, in one implementation, when collar 132 is moved to the first activation position, the switch(es) 134 can cause the printed circuit board 138 to transmit a signal to the electrosurgical generator 110 and cause the electrosurgical generator 110 to responsively supply the electrosurgical energy with a first level of power and/or the first waveform corresponding to a mode of operation associated with the first activation position. Additionally, when collar 132 is moved to the second activation position, the switch(es) 134 can cause the printed circuit board 138 to transmit a signal to the electrosurgical generator 110 and cause the electrosurgical generator 110 to responsively supply the electrosurgical energy with a second level of power and/or the second waveform corresponding to a mode of operation associated with the second activation position.

In another implementation, moving the collar 132 to the first activation position and thereby actuating the switch(es) 134 can close the switch(es) 134 to complete a first circuit to the electrosurgical generator 110 to cause the electrosurgical generator 110 to responsively supply the electrosurgical energy with the first level of power and/or the first waveform corresponding to the mode of operation associated with the first activation position. In an example of this implementation including the second activation position, moving the collar 132 to the second activation position and thereby actuating the switch(es) 134 can close the switch(es) 134 to complete a second circuit to the electrosurgical generator 110 to cause the electrosurgical generator 110 to responsively supply the electrosurgical energy with the second level of power and/or the second waveform corresponding to a mode of operation associated with the second activation position. In some examples of this implementation, the printed circuit board 138 can be omitted.

In both example implementations, the electrosurgical energy supplied by the electrosurgical generator 110 can be supplied from (i) the power cord 122, the printed circuit board 138, and/or the switch(es) 134 to (ii) the electrosurgical electrode 128 by the housing conductor 140 and the shaft conductor 142. As such, as shown in FIG. 1, the printed circuit board 138 can be coupled to the power cord 122, the housing conductor 140 can be coupled to the printed circuit board 138 and the shaft conductor 142, and the shaft conductor 142 can be coupled to the electrosurgical electrode 128. In this arrangement, the housing conductor 140 can conduct the electrosurgical energy (supplied to the housing conductor 140 via the printed circuit board 138) to the shaft conductor 142, and the shaft conductor 142 can conduct the electrosurgical energy to the electrosurgical electrode 128. The switch(es) 134 can be coupled to the printed circuit board 138 in some examples.

In general, the housing conductor 140 and the shaft conductor 142 can each include one or more electrically conductive elements that provide an electrically conductive bus for supplying the electrosurgical energy to the electrosurgical electrode 128. More particularly, the housing conductor 140 can include one or more electrically conductive elements of the handle 124 that can supply the electrosurgical energy to the shaft conductor 142, and the shaft conductor 142 can include one or more electrically conductive elements of the shaft 126 that can supply the electrical energy from the housing conductor 140 to the electrosurgical electrode 128. In some examples, the housing conductor 140 and the shaft conductor 142 can be coupled to each other in a manner that is suitable to maintain an electrical coupling between the housing conductor 140, the shaft conductor 142, and the electrosurgical electrode 128 while (i) the shaft 126 and/or the electrosurgical electrode 128 telescopically moves relative to the handle 124, and/or (ii) the electrosurgical electrode 128 rotates relative to the handle 124.

As noted above, the electrosurgical electrode 128 can apply the electrosurgical energy to a target tissue to perform an electrosurgical operation (e.g., cutting, coagulating, ablating, and/or sealing the target tissue). Within examples, the electrosurgical electrode 128 can include an electrosurgical substrate formed from an electrically conductive material. As an example, the electrically conductive material can be stainless steel.

The electrosurgical substrate can extend in an axial direction from a proximal end of the electrosurgical electrode 128 to a distal end of the electrosurgical electrode 128. The proximal end of the electrosurgical electrode 128 can receive electrosurgical energy from the electrosurgical instrument 112 (e.g., via the housing conductor 140 and the shaft conductor 142 as described above), and a distal working portion of the electrosurgical electrode 128 can apply the electrosurgical energy to the target tissue. In one implementation, the electrosurgical substrate can include a shank portion that extends from the proximal end of electrosurgical electrode 128 to the distal working portion of the electrosurgical electrode 128. The distal working portion can be configured to use the electrosurgical energy to at least one of cut or coagulate tissue in a monopolar electrosurgical operation.

In some examples, the distal working portion can define an electrosurgical blade. For instance, the electrosurgical blade can include (i) a first lateral surface, (ii) a second lateral surface opposite the first lateral surface, (iii) a first major surface extending between the first lateral surface and the second lateral surface on a first side of the electrosurgical blade, and (iv) a second major surface extending between the first lateral surface and the second lateral surface on a second side of the electrosurgical blade that is opposite the first side. The first lateral surface and the second lateral surface have surface areas that are relatively small compared to surface areas of the first major surface and the second major surface such that a thickness (e.g., a dimension between the first major surface and the second major surface) of the electrosurgical blade is relatively small as compared to a length (e.g., a dimension extending between the proximal end and the distal end of the electrosurgical electrode 128) and a width (e.g., a dimension between the first latera surface and the second lateral surface).

In some examples, the distal working portion of the electrosurgical electrode 128 can also include an outer layer of material covering at least a portion (or an entirety) of the electrosurgical substrate. For instance, the outer layer of material can be formed from at least one material selected from a group consisting of: a polymeric material, a fluorocarbon material (e.g., polytetrafluoroethylene (PTFE)), silicone, enamel, a ceramic material, and inorganic lubricant material (e.g., titanium nitride, zirconium nitride, titanium aluminum nitride, and nitron). The outer layer of material can help to, for example, inhibit eschar build-up and/or focus the electrosurgical energy to one or more portions of the electrosurgical electrode 128.

In some examples, the distal working portion of the electrosurgical electrode 128 can additionally include an intermediate layer between the electrosurgical substrate and the outer layer. The intermediate layer can be configured to provide thermal conductivity to help mitigate heating of the outer layer leading to a breakdown of the outer layer. The intermediate layer can also be configured to maintain the electrical conductivity of the electrosurgical substrate such that the intermediate layer does not degrade the transmission of the electrosurgical energy from the electrosurgical substrate to the target tissue.

The intermediate layer can be an anisotropic thermally conductive material, whereby the in-plane (e.g., parallel to the electrode surface) thermal conductivity substantially exceeds the out-of-plane (e.g., perpendicular to the electrode surface) thermal conductivity. The anisotropic thermally conductive material having a coefficient of thermal expansion matched (or approximately 10% greater or approximately 10% lower) to the electrosurgical substrate and outer layer. As an example, this intermediate layer can include at least one material selected from a group consisting of pyrolytic graphite/carbon, graphene, and Molybdenum disulfide.

As shown in FIG. 1, in some implementations, the electrosurgical instrument 112 can additionally include one or more light sources 144 that are configured to emit light. In some examples that include the light source(s) 144, the user activation device 130 can be operable to cause the light source(s) 144 to generate light that can be emitted by the electrosurgical instrument 112 to illuminate an area of interest (e.g., a target tissue at the surgical site). In some implementations, the light source(s) 144 can be located at a distal end of the housing 123 and/or a distal end of the shaft 126 to directly provide light in a distal direction and illuminate a surgical distal of the electrosurgical electrode 128.

In other implementations, as shown in FIG. 1, the light source(s) 144 can be optically coupled to an optical structure 146, which is configured to receive the light emitted by the light source(s) 144 and transmit the light in a distal direction toward a surgical site to illuminate the surgical site while performing electrosurgery using the electrosurgical electrode 128. Although arranging the light source(s) 144 to directly illuminate a surgical field can help, for instance, to reduce a cost of manufacture, transmitting the light using the optical structure 146 can help to improve a quality of light transmitted from the electrosurgical instrument 112 (e.g., by providing light with improved uniformity and/or reduced heat generation).

As examples, in implementations that include the optical structure 146, the optical structure 146 can include at least one optical structure selected from among a group consisting of an optical lens, a non-fiber optic optical waveguide, and an optical fiber. When the optical structure 146 includes the optical lens (e.g., a parabolic reflector lens, an aspheric lens, and/or a Fresnel lens), the optical structure 146 can help to direct the light emitted by the light source 144 in the distal direction and thereby improve a quality of the light illuminating the surgical site. The optical structure 146 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 146 can additionally or alternatively include other light shaping optical elements such as, for instance, a plurality of facets, one or more prisms, and/or one or more optical gratings. Although the optical structure 146 can help to improve a quality of the light directed to the surgical site, the electrosurgical instrument 112 can omit the optical structure 146 and instead emit the light from the light source 144 directly to the surgical field without transmitting the light through the optical structure 146 in other examples.

In FIG. 1, the light source 144 can be coupled to the shaft 126. As such, the light source 144 can also move telescopically with the shaft 126 relative to the handle 124. However, in other examples, the light source 144 can be in the interior bore of the handle 124 and/or coupled to an exterior surface of the handle 124. As examples, the light source 144 can include one or more light emitting diodes (LEDs), organic light emitting diodes (OLEDs), optical fibers, non-fiber optic waveguides, and/or lenses. Additionally, for example, the light source 144 can include a LED printed circuit board having one or more light sources (e.g., LEDs).

The optical structure 146 can be at a distal end of the shaft 126. In some examples, the optical structure 146 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical instrument 112 relative to the target tissue. However, in other examples, the optical structure 146 can extend partially but not fully around the electrosurgical electrode 128.

In implementations that include the light source 144, the user activation device(s) 130, the printed circuit board 138, the switch(e)s 134, the housing conductor 140, and/or the shaft conductor 142 can additionally supply an electrical power from a direct current (DC) power source 148 to the light source 144. In one example, the DC power source 148 can include a battery disposed in the handle 124, the plug of the power cord 122, and/or a battery receptacle located along the power cord 122 between the handle 124 and the plug. Although the electrosurgical instrument 112 includes the DC power source 148 in FIG. 1, the DC power source 148 can be separate and distinct from the electrosurgical instrument 112 in other examples. For instance, in another example, the electrosurgical generator 110 can include the DC power source 148.

Additionally, in implementations that include the light source 144, the user activation device(s) 130 can be operable to cause the light source 144 to emit the light. For instance, in one example, moving the collar 132 to the first activation position can cause (i) the electrosurgical generator 110 to supply the electrosurgical energy with the first waveform to the electrosurgical electrode 128 and (ii) the DC power source 148 to supply DC power to the light source(s) 144. This can provide for simultaneous illumination and electrosurgical operation. Additionally, in this example, moving the collar 132 to the second activation position can cause the electrosurgical generator 110 to supply the electrosurgical energy with the first waveform to the electrosurgical electrode 128 without the DC power source 148 supplying DC power to the light source(s) 144. Therefore, in this example, the same waveform of electrosurgical energy can be supplied in both the first activation position and the second activation position, but light can be emitted in only the first activation position and not the second activation position.

In another example, moving the collar 132 to the first activation position can cause (i) the electrosurgical generator 110 to supply the electrosurgical energy with the first waveform to the electrosurgical electrode 128 and (ii) the DC power source 148 to supply DC power to the light source(s) 144. Additionally, in this example, moving the collar 132 to the second activation position can cause (i) the electrosurgical generator 110 to supply the electrosurgical energy with the second waveform to the electrosurgical electrode 128 and (ii) the DC power source 148 to supply DC power to the light source(s) 144. Therefore, in this example, the user activation device(s) 130 cause light to be emitted in both the first activation position and the second activation, but with different waveforms of the electrosurgical energy supplied.

In yet another example, the user activation device(s) 130 can include a button that independently controls the light source(s) 144 separate from the collar 132 and the switch(es) 134 that control the electrosurgical operational modes of the electrosurgical instrument 112.

As shown in FIG. 1, responsive to operation of the user activation device(s) 130 to actuate the light source 144, the DC power source 148 can supply the electrical power (e.g., a DC voltage) to the light source(es) 144 via the printed circuit board 138, the housing conductor 140, and/or the shaft conductor 142. In this implementation, one or more of the conductive elements of the housing conductor 140 can be configured to supply the electrical power from the DC power source 148 to the light source(s) 144 and/or return the electrical power from the light source(s) 144 to the DC power source 148. Accordingly, the housing conductor 140 can additionally or alternatively assist in providing electrical communication between the DC power source 148 and the light source(s) 144 as the shaft 126 and the light source(s) 144 telescopically move relative to the handle 124.

Although the user activation device(s) 130 on the handle 124 can be operated to control the operation of the light source(s) 144 in the examples described above, the light source(s) 144 can be additionally or alternatively operated by one or more user activation device(s) on the electrosurgical generator 110 (e.g., via the user interface 116) and/or on the plug of the power cord 122.

In some examples, the electrosurgical instrument 112 can additionally or alternatively include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 150 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 150 and the electrosurgical generator 110 can be provided as separate devices or integrated in a single device (e.g., in a common housing).

As shown in FIG. 1, the shaft 126 can include a smoke evacuation channel 152 in the inner cavity of the shaft 126. The smoke evacuation channel 152 can also include a smoke inlet that can extend circumferentially around a center axis of a distal portion of the electrosurgical electrode 128. In this arrangement, the smoke inlet of the smoke evacuation channel can help to receive surgical smoke into the smoke evacuation channel 152 in all rotational alignments of the electrosurgical electrode 128 relative to the handle 124 and/or the electrosurgical instrument 112 relative to the target tissue. However, in another example, the smoke evacuation channel 152 can include one or more smoke inlets that do not extend circumferentially around the electrosurgical electrode 128.

In an example, the smoke evacuation channel 152 of the shaft 126 defines a first portion of a smoke flow path, and the interior bore 125 of the handle 124 defines a second portion of a smoke flow path. In this arrangement, the surgical smoke can be received from the surgical site into the smoke evacuation channel 152 of the shaft 126, and flow proximally along the smoke evacuation channel 152 to the interior bore 125 of the handle 124. In the interior bore 125 of the handle 124, the smoke can further flow to a smoke tube 154 that is coupled to a proximal end of the handle 124 and configured to convey smoke from the handle 124 to the suction pump 150.

Additionally, in some implementations that provide for surgical smoke evacuation, the user activation device(s) 130 can be operable to cause the suction pump 150 to generate suction. For instance, in one example, moving the collar 132 to the first activation position can cause (i) the electrosurgical generator 110 to supply the electrosurgical energy with the first waveform to the electrosurgical electrode 128 and (ii) the suction pump 150 to generate suction. This can provide for simultaneous smoke evacuation and electrosurgical operation. Additionally, in this example, moving the collar 132 to the second activation position can cause the electrosurgical generator 110 to supply the electrosurgical energy with the first waveform to the electrosurgical electrode 128 without the suction pump 150 generating suction. Therefore, in this example, the same waveform of electrosurgical energy can be supplied in both the first activation position and the second activation position, but suction can be generated in only the first activation position and not the second activation position.

In another example, moving the collar 132 to the first activation position can cause (i) the electrosurgical generator 110 to supply the electrosurgical energy with the first waveform to the electrosurgical electrode 128 and (ii) the suction pump 150 to generate suction. Additionally, in this example, moving the collar 132 to the second activation position can cause (i) the electrosurgical generator 110 to supply the electrosurgical energy with the second waveform to the electrosurgical electrode 128 and (ii) the suction pump 150 to generate suction. Therefore, in this example, the suction pump 150 can generate suction in both the first activation position and the second activation, but with different waveforms of the electrosurgical energy supplied.

In other examples, the suction pump 150 can be activated and/or deactivated responsive to a user input on the suction pump 150 and/or responsive to a sensor signal. For instance, a sensor can be positioned along the smoke flow path and configured to generate the sensor signal responsive to the sensor detecting a presence and/or greater than a threshold amount of smoke.

FIGS. 2A-2C depict an implementation of the electrosurgical instrument 112 shown and described above with respect to FIG. 1, according to an example. In particular, FIG. 2A depicts the electrosurgical instrument 112 with the collar 132 in the home position, FIG. 2B depicts the electrosurgical instrument 112 with the collar 132 in the first activation position, and FIG. 2C depicts the electrosurgical instrument 112 in the second activation position, according to the example.

As shown in FIGS. 2A-2C, the housing 123 extends from a proximal end 223A to a distal end 223B, and the electrosurgical electrode 128 extends from the distal end 223B of the housing 123. The user activation device 130 disposed between the proximal end 223A of the housing 123 and the distal end 223B of the housing 123, and the user activation device 130 is operable to control a supply of electrosurgical energy to the electrosurgical electrode 128.

In this example, the user activation device 130 includes the collar 132 that extends around an entirety of the circumference of the housing 123. However, as described above, the collar 132 can extend around at least a half of a circumference of the housing 123 in other examples (e.g., the collar 132 can extend around a portion of the circumference of the housing 123 that is between 50 percent to 100 percent of the circumference of the housing 123). Also, in FIGS. 2A-2C, the housing 123 can have a circular cross-sectional shape or a non-circular cross-sectional shape.

As shown in FIGS. 2A-2C, the user activation device 130 also includes the switch 134 configured to control the supply of the electrosurgical energy to the electrosurgical electrode responsive to the collar 132 moving from the home position (shown in FIG. 2A) to the first activation position (shown in FIG. 2B) and/or the second activation position (shown in FIG. 2C), as described above. In this example, the switch 134 includes a rocker switch that is mechanically actuatable between a first position (shown in FIG. 2A), a second position (shown in FIG. 2B), and a third position (shown in FIG. 2C). The collar 132 is configured to slide over and mechanically engage the switch 134 such that (i) the switch 134 is in the first position when the collar 132 is in the home position as shown in FIG. 2A, (ii) the switch 134 is in the second position when the collar 132 is in the first activation position as shown in FIG. 2B, and (iii) the switch 134 is in the third position when the collar 132 is in the second activation position as shown in FIG. 2C.

For instance, the rocker switch can include a rocker paddle 234A, a first contact 234B, and a second contact 234C. When the switch 134 is in the first position corresponding to the home position of the collar 132, the rocker paddle 234A does not depress the first contact 234B or the second contact 234C as shown in FIG. 2A. When the switch 134 is in the second position corresponding to the first activation position of the collar 132, the rocker paddle 234A depresses the first contact 234B as shown in FIG. 2B. When the switch 134 is in the third position corresponding to the second activation position of the collar 132, the rocker paddle 234A depresses the second contact 234C as shown in FIG. 2C. Because the collar 132 extends around circumference of the housing 123, the switch 134 can be operated between the first position, the second position, and the third position in any rotational orientation of the housing 123 relative to the hand of the user and regardless of a position of the hand of the user relative to the switch 134 underlying the collar 132.

Additionally, in the example shown in FIGS. 2A-2C, the user activation device 130 includes the biasing member 136 that biases the collar 132 towards the home position. In particular, in this example, the biasing member 136 includes one or more springs that bias the rocker paddle 234A towards the first position shown in FIG. 2A.

In this arrangement, when the collar 132 is in the home position relative to the housing 123 shown in FIG. 2A, the switch 134 is in the first position and the switch 134 and/or the printed circuit board 138 prevent the electrosurgical energy from being supplied to the electrosurgical electrode 128. When the collar 132 is moved from the home position shown in FIG. 2A to the first activation position shown in FIG. 2B (e.g., against a biasing force applied by the biasing member 136), the collar 132 mechanically contacts and forces rocker paddle 234A to depress the first contact 234B. As a result, the switch 134 and/or the printed circuit board 138 cause the electrosurgical electrode 128 to receive the electrosurgical energy with the first level of power and/or the first waveform (and/or causes the light source 144 to emit light and/or causes the suction pump 150 to generate suction). Responsive to the user releasing the collar 132 at the first activation position, the biasing member 136 forces the collar 132 back to the home position and the switch 134 back to the first position.

When the collar 132 is moved from the home position shown in FIG. 2A to the second activation position shown in FIG. 2C (e.g., against a biasing force applied by the biasing member 136), the collar 132 mechanically contacts and forces rocker paddle 234A to depress the second contact 234C. As a result, the switch 134 and/or the printed circuit board 138 cause the electrosurgical electrode 128 to receive the electrosurgical energy with the second level of power and/or the second waveform (and/or causes the light source 144 to emit light and/or causes the suction pump 150 to generate suction). Responsive to the user releasing the collar 132 at the second activation position, the biasing member 136 forces the collar 132 back to the home position and the switch 134 back to the first position.

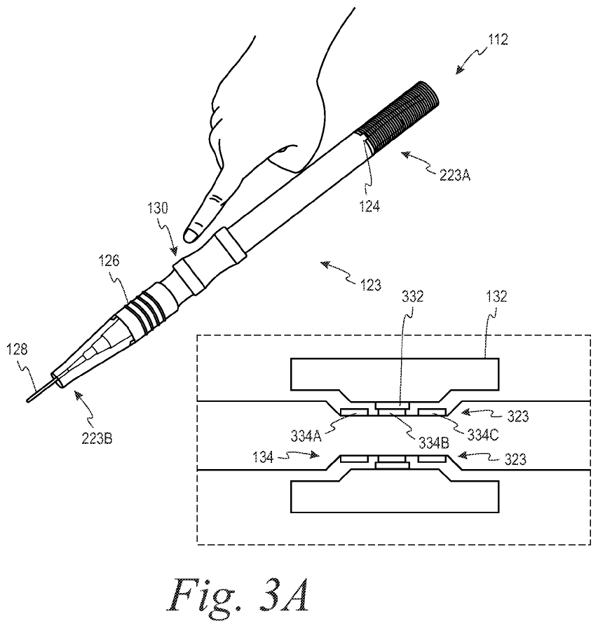

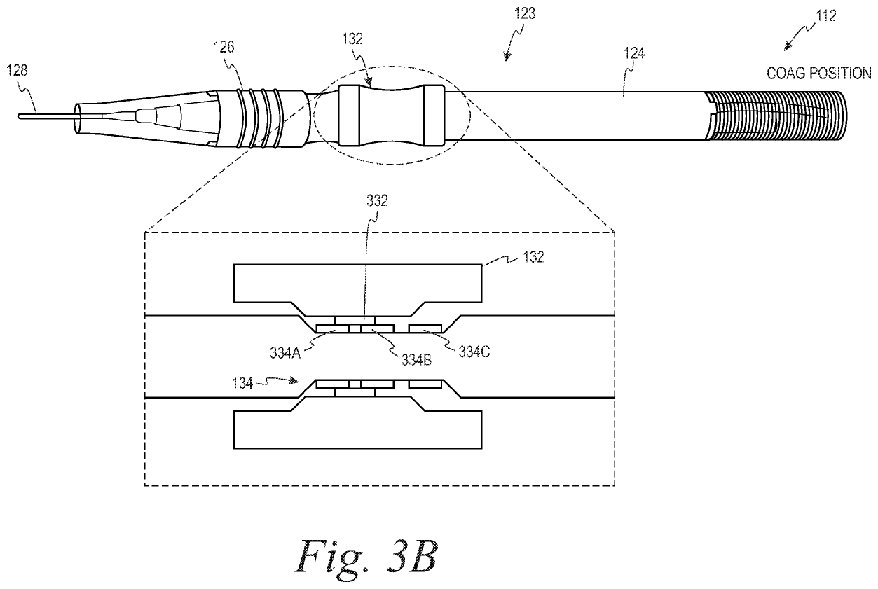

FIGS. 3A-3C depict an implementation of the electrosurgical instrument 112 shown and described above with respect to FIG. 1, according to another example. In particular, FIG. 3A depicts the electrosurgical instrument 112 with the collar 132 in the home position, FIG. 3B depicts the electrosurgical instrument 112 with the collar 132 in the first activation position, and FIG. 3C depicts the electrosurgical instrument 112 in the second activation position, according to the example.

As shown in FIGS. 3A-3C, the housing 123 extends from a proximal end 223A to a distal end 223B, and the electrosurgical electrode 128 extends from the distal end 223B of the housing 123. The user activation device 130 disposed between the proximal end 223A of the housing 123 and the distal end 223B of the housing 123, and the user activation device 130 is operable to control a supply of electrosurgical energy to the electrosurgical electrode 128.

In this example, the user activation device 130 includes the collar 132 that extends around an entirety of the circumference of the housing 123. However, as described above, the collar 132 can extend around at least a half of a circumference of the housing 123 in other examples (e.g., the collar 132 can extend around a portion of the circumference of the housing 123 that is between 50 percent to 100 percent of the circumference of the housing 123). Also, in FIGS. 3A-3C, the housing 123 can have a circular cross-sectional shape or a non-circular cross-sectional shape.

As shown in FIGS. 3A-3C, the user activation device 130 also includes the switch 134 configured to control the supply of the electrosurgical energy to the electrosurgical electrode responsive to the collar 132 moving from the home position (shown in FIG. 3A) to the first activation position (shown in FIG. 3B) and/or the second activation position (shown in FIG. 3C), as described above. In this example, the switch 134 includes a first electrical contact 334A, a second electrical contact 334B, and a third electrical contact 334C. The first electrical contact 334A, the second electrical contact 334B, and the third electrical contact 334C can be on an exterior surface of the housing 123. An electrical potential can be applied to one or more of the first electrical contact 334A, the second electrical contact 334B, and the third electrical contact 334C. As shown in FIG. 3A, the first electrical contact 334A, the second electrical contact 334B, and the third electrical contact 334C can be separated from each other by respective gaps (e.g., not in direct electrical communication with each other).

Additionally, in this example, the collar 132 includes one or more conductive elements 332 (e.g., one or more conductive pads) that are configured to electrical contact one or more of the first electrical contact 334A, the second electrical contact 334B, and the third electrical contact 334C at a given time. The conductive elements 332 can be on an inner surface of the collar 132 (e.g., facing the first electrical contact 334A, the second electrical contact 334B, and the third electrical contact 334C).

As shown in FIG. 3A, when the collar 132 is in the home position, the conductive element(s) 332 of the collar 132 contact less than two of the first electrical contact 334A, the second electrical contact 334B, and the third electrical contact 334C such that an open circuit condition exists between the first electrical contact 334A, the second electrical contact 334B, and the third electrical contact 334C. In this state, the switch 134 can prevent the electrosurgical electrode 128 from receiving the electrosurgical energy.

As shown in FIG. 3B, when the collar 132 is in the first activation position, the one or more conductive elements 332 electrically contact the first electrical contact 334A and the second electrical contact 334B. As such, the conductive element(s) 332 can close a first circuit including the first electrical contact 334A and the second electrical contact 334B. In one example, this result in the electrical potential being provided to the printed circuit board 138 to provide a signal for supplying the electrosurgical energy with the first level of power and/or the first waveform.

As shown in FIG. 3C, when the collar 132 is in the second activation position, the one or more conductive elements 332 electrically contact the second electrical contact 334B and the third electrical contact 334C. As such, the conductive element(s) 332 can close a second circuit including the second electrical contact 334B and the third electrical contact 334C. In one example, this result in the electrical potential being provided to the printed circuit board 138 to provide a signal for supplying the electrosurgical energy with the second level of power and/or the second waveform.

Because the collar 132 extends around circumference of the housing 123, the switch 134 can be operated to selectively close the first circuit, close the second circuit, or open both the first circuit and the second circuit any rotational orientation of the housing 123 relative to the hand of the user and regardless of a position of the hand of the user relative to the switch 134 underlying the collar 132.

Additionally, as shown in FIGS. 3A-3B, the housing 123 includes a recessed portion 323 that has cross-sectional dimensions that are less than cross-sectional dimensions of the housing 123 on opposing sides of the recessed portion 323 (e.g., in a dimension that is perpendicular to a longitudinal axis of the housing 123). The collar is disposed and movable in the recessed portion 323 of the housing 123. The recessed portion 323 can help to limit a range of movement of the collar 132 relative to the housing 123. For instance, the recessed portion 323 can define a distal stop and a proximal stop that limit the range of movement of the collar 132. Additionally or alternatively, the recessed portion 323 can have a sloped distal wall and a sloped proximal wall, which can engage correspondingly sloped walls of the collar 132 at the first activation position and the second activation position, respectively. The engagement of the sloped distal wall and the sloped proximal wall with the correspondingly sloped walls of the collar 132 can assist in biasing the collar 132 towards the home position.

In FIGS. 3A-3C, the collar 132 has cross-sectional dimensions of the collar 132 can be greater than the cross-sectional dimensions of the housing 123 on opposing sides of the recessed portion 323 such that the collar 132 protrudes outwardly from the exterior surface of the housing 123. This can help with ease of manufacture and assembly. However, in another example, the collar 132 and the recessed portion 323 of the housing 123 can be configured such that an outer surface of the collar 132 is approximately flush with the exterior surface of the housing 123 on opposing sides of the recessed portion 323. This can help to reduce the overall cross-sectional dimensions of the electrosurgical instrument 112, which may provide for enhanced lines of sight to the surgical site.

Although the housing 123 includes the recessed portion 323 in FIGS. 3A-3C, the housing 123 can omit the recessed portion 323 in other examples. For instance, the housing 123 can have the same cross-sectional dimensions at each point along the range of movement of the collar 132 as well as on opposing sides of the range of movement of the collar 132.

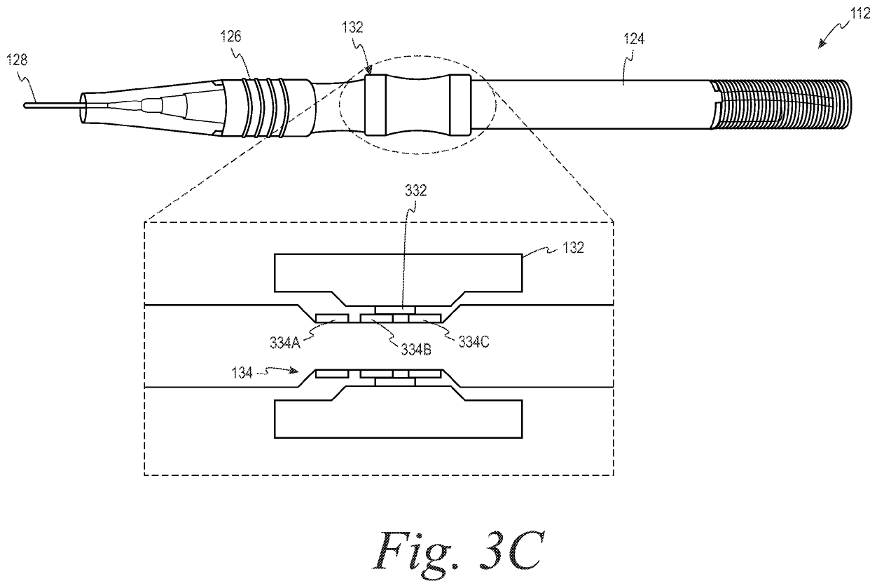

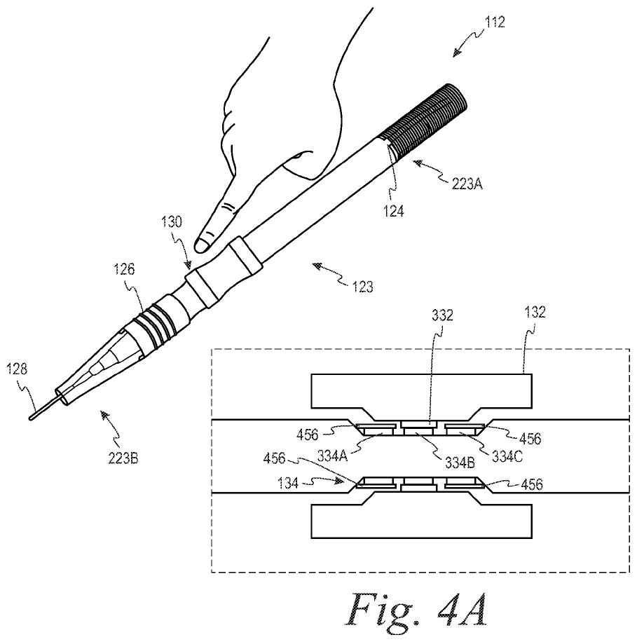

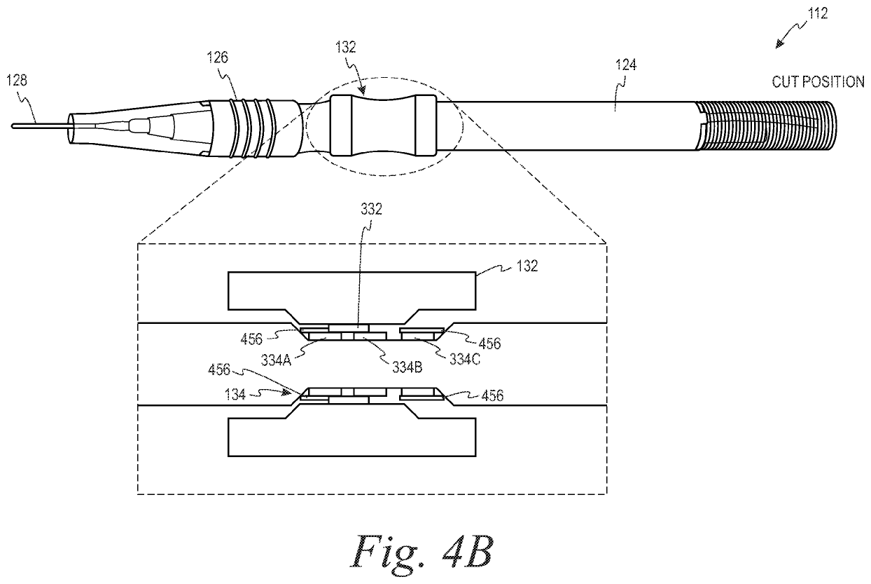

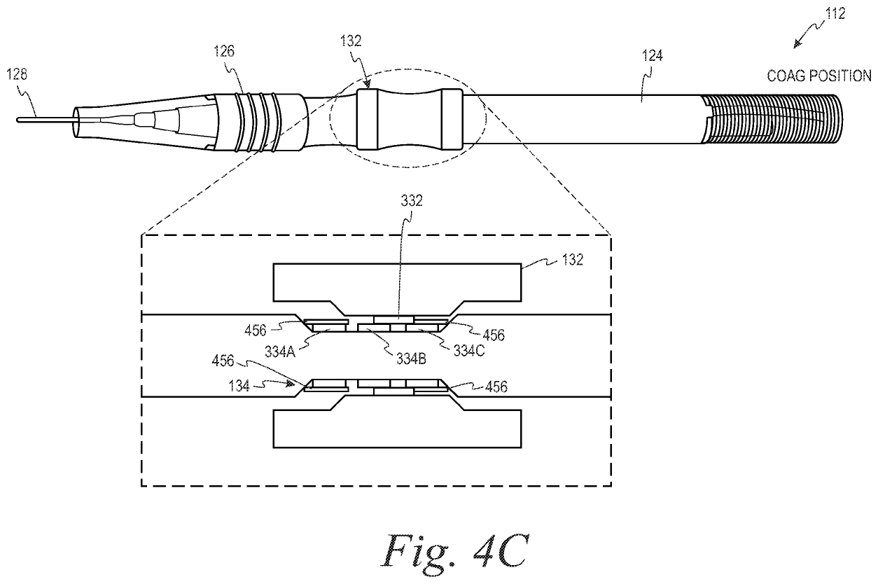

FIGS. 4A-4C depict an implementation of the electrosurgical instrument 112 shown and described above with respect to FIG. 1, according to another example. In particular, FIG. 4A depicts the electrosurgical instrument 112 with the collar 132 in the home position, FIG. 4B depicts the electrosurgical instrument 112 with the collar 132 in the first activation position, and FIG. 4C depicts the electrosurgical instrument 112 in the second activation position, according to the example.

As shown in FIGS. 4A-4C, the housing 123 extends from a proximal end 223A to a distal end 223B, and the electrosurgical electrode 128 extends from the distal end 223B of the housing 123. The user activation device 130 disposed between the proximal end 223A of the housing 123 and the distal end 223B of the housing 123, and the user activation device 130 is operable to control a supply of electrosurgical energy to the electrosurgical electrode 128.

In this example, the user activation device 130 is substantially similar or identical to the user activation device 130 described above with respect to FIGS. 3A-3C, except the switch 134 in FIGS. 4A-4C also include a plurality of flaps 456 covering respective ones of the first electrical contact 334A and the third electrical contact 334C. The flaps 456 can be formed from a material that is an electrical insulator to inhibit or prevent electrical transmission through the flaps 456. The collar 132 can be configured to move the plurality of flaps 456 to expose the respective ones of the first electrical contact 334A and the third electrical contact 334C responsive to the collar 132 moving relative to the housing 123.

In this arrangement, when the collar 132 is in the home position shown in FIG. 4A, the flaps 456 cover the first electrical contact 334A and the third electrical contact 334C. The flaps 456 can further assist in inhibiting electrical coupling between the first electrical contact 334A, the second electrical contact 334B, and the third electrical contact 334C when the collar 132 is in the home position (e.g., in the event of liquid ingress into a space between the collar 132 and the electrical contacts 334A-334C).

As shown in FIG. 4B, when the collar 132 moves to the first activation position, the collar 132 and/or the conductive element 332 can engage and move the flap 456 over the first electrical contact 334A such that the first electrical contact 334A is exposed. As a result, when the collar 132 is in the first activation position, the conductive element(s) 332 can electrically couple the first electrical contact 334A and the second electrical contact 334B as described above.

As shown in FIG. 4C, when the collar 132 moves to the second activation position, the collar 132 and/or the conductive element 332 can engage and move the flap 456 over the third electrical contact 334C such that the third electrical contact 334C is exposed. As a result, when the collar 132 is in the second activation position, the conductive element(s) 332 can electrically couple the second electrical contact 334B and the third electrical contact 334C as described above.

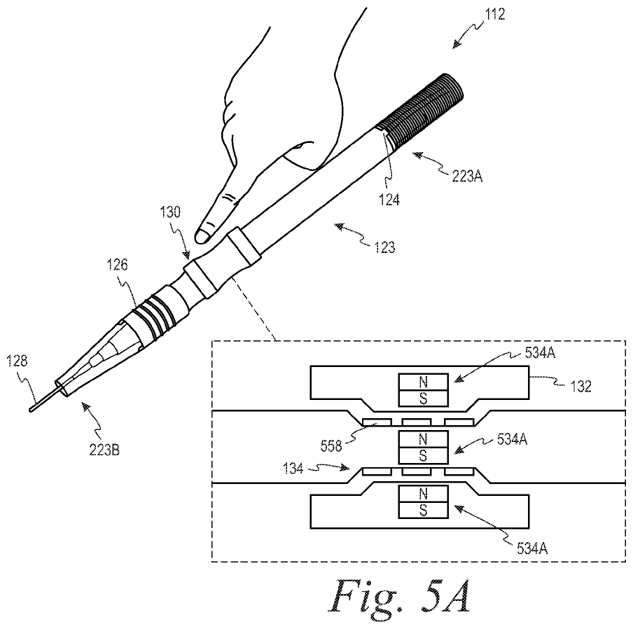

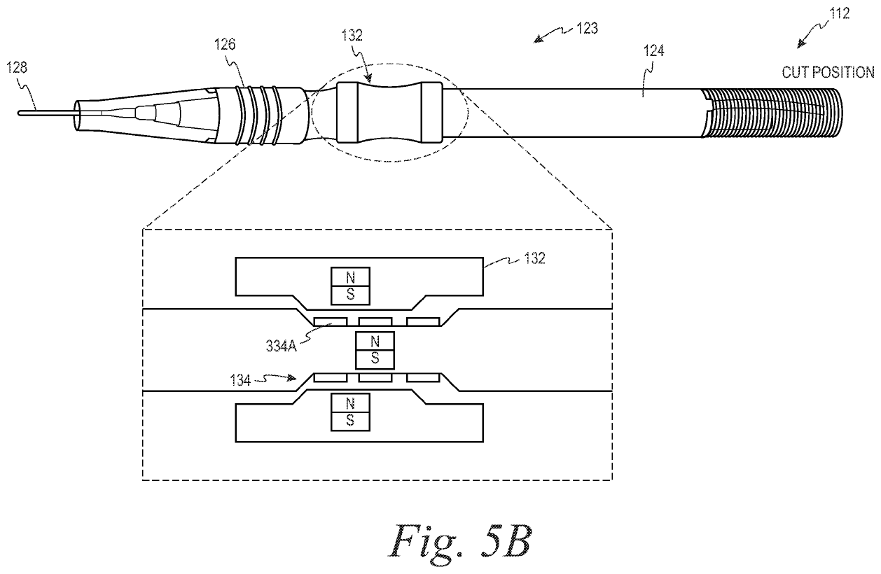

FIGS. 5A-5C depict an implementation of the electrosurgical instrument 112 shown and described above with respect to FIG. 1, according to another example. In particular, FIG. 3A depicts the electrosurgical instrument 112 with the collar 132 in the home position, FIG. 3B depicts the electrosurgical instrument 112 with the collar 132 in the first activation position, and FIG. 3C depicts the electrosurgical instrument 112 in the second activation position, according to the example.

As shown in FIGS. 5A-5C, the housing 123 extends from a proximal end 223A to a distal end 223B, and the electrosurgical electrode 128 extends from the distal end 223B of the housing 123. The user activation device 130 disposed between the proximal end 223A of the housing 123 and the distal end 223B of the housing 123, and the user activation device 130 is operable to control a supply of electrosurgical energy to the electrosurgical electrode 128.

In this example, the user activation device 130 includes the collar 132 that extends around an entirety of the circumference of the housing 123. However, as described above, the collar 132 can extend around at least a half of a circumference of the housing 123 in other examples (e.g., the collar 132 can extend around a portion of the circumference of the housing 123 that is between 50 percent to 100 percent of the circumference of the housing 123). Also, in FIGS. 5A-5C, the housing 123 can have a circular cross-sectional shape or a non-circular cross-sectional shape.

As shown in FIGS. 5A-5C, the user activation device 130 also includes the switch 134 configured to control the supply of the electrosurgical energy to the electrosurgical electrode responsive to the collar 132 moving from the home position (shown in FIG. 3A) to the first activation position (shown in FIG. 3B) and/or the second activation position (shown in FIG. 3C), as described above. In this example, the switch 134 includes a Hall Effect sensor 558 that is configured to sense when the collar 132 is in the home position, when the collar 132 is in the first activation position, and when the collar 132 is in the second activation position.

For instance, in the example shown in FIGS. 5A-5C, the collar 132 can include a first magnet 560 and the housing 123 can include a second magnet 562 such that the first magnet 560 and the second magnet 562 interact to form a magnetic field at a position of the Hall Effect sensor 558. In FIG. 5A, when the collar 132 is in the home position, the magnetic field can have a first magnitude and a first direction based on the relative positions of the first magnet 560 and the second magnet 562. The Hall Effect sensor 558 can sense the magnetic field having the first magnitude and the first direction and responsively signal to the printed circuit board 138 (shown in FIG. 1) that the collar 132 is in the home position. In this way, the Hall Effect sensor 558 can cause the printed circuit board 138 to prevent the electrosurgical electrode 128 from receiving the electrosurgical energy.

In FIG. 5B, when the collar 132 is in the first activation position, the magnetic field can have a second magnitude and a second direction based on the relative positions of the first magnet 560 and the second magnet 562. The Hall Effect sensor 558 can sense the magnetic field having the second magnitude and the second direction and responsively signal to the printed circuit board 138 (shown in FIG. 1) that the collar 132 is in the first activation position. In this way, the Hall Effect sensor 558 can cause the printed circuit board 138 to cause supplying the electrosurgical energy to the electrosurgical electrode 128 with the first level of power and/or the first waveform according to one example.

In FIG. 5C, when the collar 132 is in the second activation position, the magnetic field can have a third magnitude and a third direction based on the relative positions of the first magnet 560 and the second magnet 562. The Hall Effect sensor 558 can sense the magnetic field having the third magnitude and the third direction and responsively signal to the printed circuit board 138 (shown in FIG. 1) that the collar 132 is in the second activation position. In this way, the Hall Effect sensor 558 can cause the printed circuit board 138 to cause supplying the electrosurgical energy to the electrosurgical electrode 128 with the second level of power and/or the second waveform according to one example.

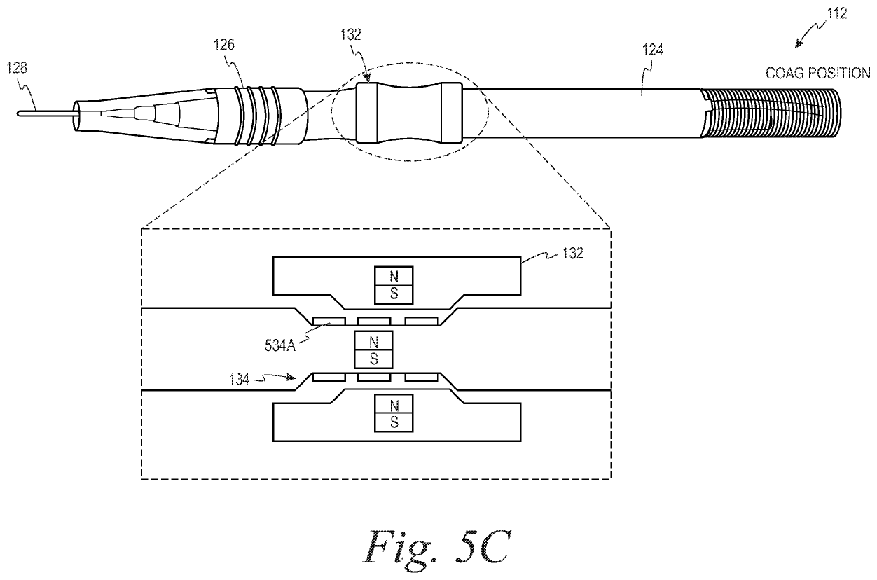

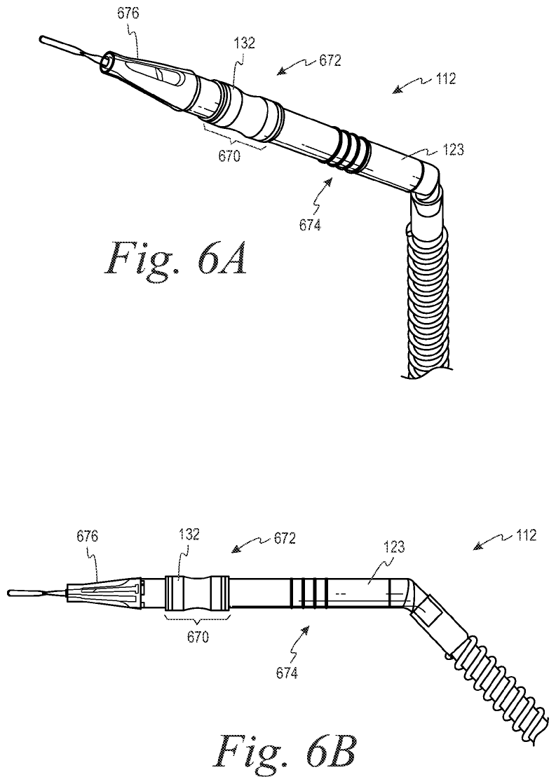

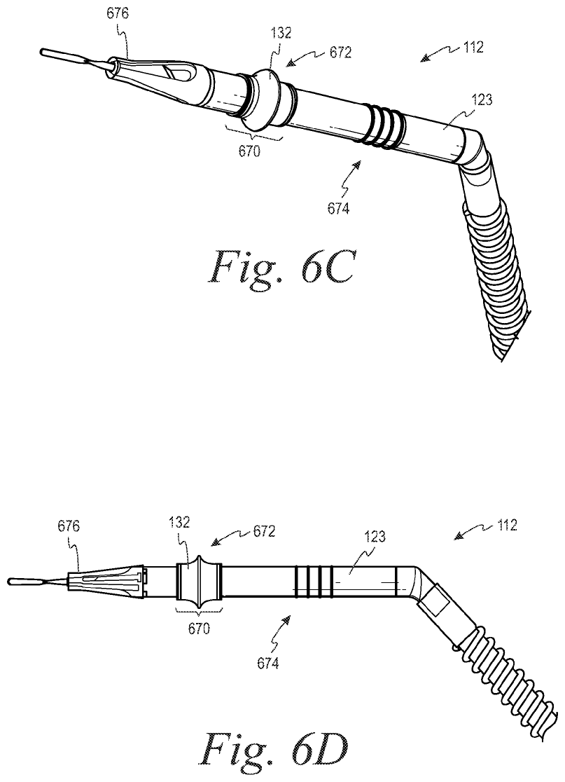

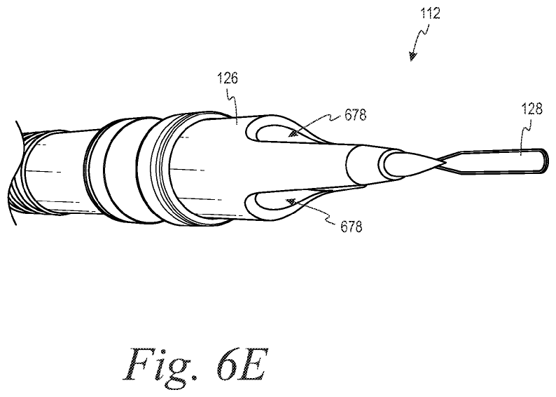

FIGS. 6A-6D depict two implementations of the electrosurgical instrument 112 shown and described above with respect to FIG. 1, according to further examples. In particular, FIG. 6A depicts a perspective view of a first example implementation of the electrosurgical instrument 112, FIG. 6B depicts a side view of the electrosurgical instrument 112 shown in FIG. 6A, FIG. 6C depicts a perspective view of a second example implementation of the electrosurgical instrument 112, and FIG. 6D depicts a side view of the electrosurgical instrument 112 shown in FIG. 6C.

In FIGS. 6A-6B, an outer surface of a central portion 670 of the collar 132 has a cross-sectional dimension (e.g., through a plane that is perpendicular to a longitudinal axis of the handle 124) that is less than a cross-sectional dimension of a distal portion and a proximal portion of the collar 132. For instance, the central portion 670 of the collar can have a concave shape. This can help to reduce the overall cross-sectional dimensions of the electrosurgical instrument 112, which may provide for enhanced lines of sight to the surgical site. By contrast, in FIGS. 6C-6D, the outer surface of the central portion 670 of the collar 132 has a cross-sectional dimension (e.g., through a plane that is perpendicular to a longitudinal axis of the handle 124) that is greater than a cross-sectional dimension of the distal portion and the proximal portion of the collar 132. For instance, the central portion 670 of the collar 132 can have a convex shape. This can help to reduce strain on the practioner when moving the collar 132 in a proximal direction.

Additionally, as shown in FIGS. 6A-6D, the collar 132 can include a grip portion 672. The grip portion 672 can have a coefficient of friction that is greater than a coefficient of friction of at least a portion of the housing 123. As such, the grip portion 672 can help to mitigate a finger of the user slipping off of the collar 132 while moving the collar 132 to the first activation position and the second activation position and/or holding the collar 132 at the first activation position or the second activation position. Additionally or alternatively, the grip portion 672 can provide tactile feedback to help differentiate the collar 132 from the housing 123.

Also, as shown in FIGS. 6A-6D, the housing 123 can include a grip section 674 that is located proximal on an exterior surface of the housing 123 and proximal of the collar 132. The grip section 674 can have a coefficient of friction that is greater than a coefficient of friction of at least a portion of the housing 123. The grip section 674 can provide for enhanced grip by a portion of the hand of the user that holds the housing 123 (e.g., while a finger of the user operates the collar 132). Additionally or alternatively, the grip section 674 can help to guide the hand of the user to a preferred position of the hand on the housing 123 (e.g., on the handle 124).

FIG. 6E depicts a distal end of the shaft 126 with a suction sleeve 676 (shown in FIGS. 6A-6D) removed for illustration purposes. As shown in FIG. 6E, the shaft 126 can include a plurality of smoke inlet 678 on opposing sides of at least a portion of the electrosurgical electrode 128. Providing the shaft 126 with two smoke inlets 678 can allow each smoke inlet 678 to be relatively large relative to other examples in which the shaft 126 includes more than two smoke inlets 678. This can, for example, help to reduce clogging of the smoke inlets 678. Although the shaft 126 includes two smoke inlets 678 in FIG. 6E, the shaft 126 can include three or more smoke inlets 678 in other examples.

In the examples shown in FIGS. 6A-6D, the collar 132 is a single, monolithic structure. In other examples, the collar 132 can include a plurality of segments that each extend around a respective portion of the housing 123. In some implementations, the segments of the collar 132 can be fixed relative to each other such that moving one segment, moves all segments. This can help to reduce or minimize a quantity of the switch(es) 134.

In other implementations, the segments of the collar 132 can be movable relative to each other. This can help to reduce a strain on a finger of a user. For instance, if a user holds the electrosurgical instrument 112 with one finger above the collar 132 and another finger below the collar 132, the user may experience strain while moving the collar 132 from the home position to the first activation position and/or the second activation position due to the placement of the fingers. However, by allowing segments of the collar 132 to move relative to each other, a finger on one segment can be moved relative to a finger on another segment (e.g., which may remain stationary relative to the housing 123) to actuate the user activation device 130.

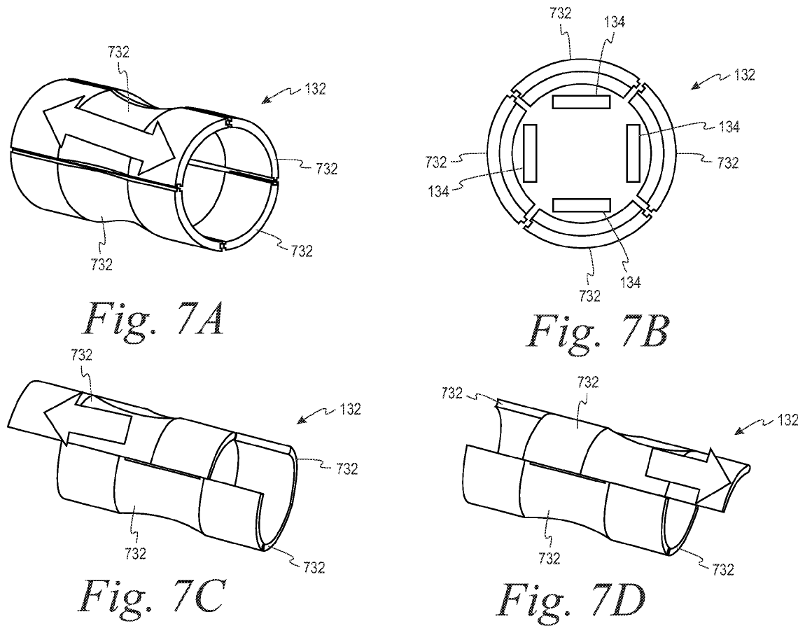

FIGS. 7A-7B depict an implementation of the collar 132 with a plurality of segments 732 that are independently movable relative to each other, according to an example. FIG. 7A depicts a perspective view of the collar 132 in the home position, FIG. 7B depicts a side view of the collar 132 and the switches 134 in the home position, FIG. 7C depicts the collar 132 in the first activation position, and FIG. 7D depicts the collar 132 in the second activation position, according to the example.

As shown in FIGS. 7A-7D, the collar 132 includes the plurality of segments 732 that are movable relative to each other, the housing 123 and the switches 134. As shown in FIG. 7B, each segment 732 is configured to actuate a respective one of the switches 134. The switches 134 can be any of the types of switches described above (e.g., mechanical switches, electrical switches, magnetic switches, piezoelectric switches, etc.).

In FIG. 7C, one segment 732 of the collar 132 has been moved to the first activation position while the other segments 732 of the collar 132 remain in the home position. With at least one segment 732 in the first activation position, the respective switch 134 is actuated to cause at least one of (i) the electrosurgical generator 110 to supply the electrosurgical energy with the first waveform to the electrosurgical electrode 128, (ii) the DC power source 148 to supply DC power to the light source(s) 144, and/or (iii) the suction pump 150 to generate suction.

In FIG. 7D, one segment 732 of the collar 132 has been moved to the second activation position while the other segments 732 of the collar 132 remain in the home position. With at least one segment 732 in the second activation position, the respective switch 134 is actuated to cause at least one of (i) the electrosurgical generator 110 to supply the electrosurgical energy with the second waveform to the electrosurgical electrode 128, (ii) the DC power source 148 to supply DC power to the light source(s) 144, and/or (iii) the suction pump 150 to generate suction.

In some examples, the segments 732 of the collar 132 can have a limited range of movement relative to the housing 123 and/or each other. For instance, each segment 732 can be disposed and movable in a recessed portion of the housing 123 (e.g., as shown and described above with respect to the recessed portion 323 and FIGS. 3A-5C).

In FIGS. 7A-7D, the collar 132 includes four segments 732. However, in other examples, the collar 132 can include two segments 732, three segments 732, or more than four segments 732.

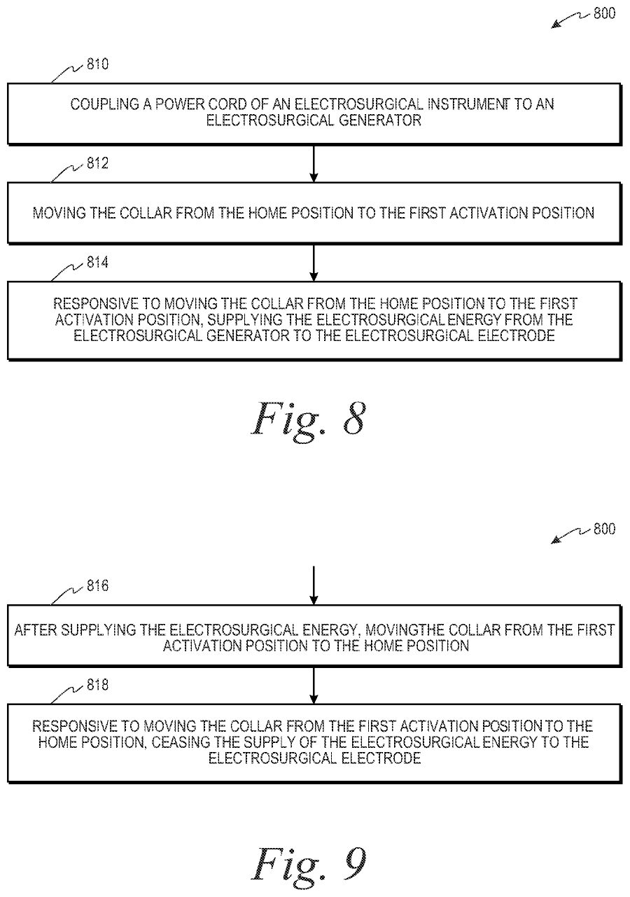

FIG. 8 depicts a flowchart for a process 800 for operating an electrosurgical instrument, according to an example. At block 810, the process 800 includes coupling a power cord of an electrosurgical instrument to an electrosurgical generator. The electrosurgical instrument includes a housing extending from a proximal end to a distal end, an electrosurgical electrode extending from the distal end of the housing, and a user activation device disposed between the proximal end of the housing and the distal end of the housing. The user activation device is operable to control a supply of electrosurgical energy to the electrosurgical electrode. The user activation device includes a collar that extends around at least a half of a circumference of the housing. The collar is movable between a home position relative to the housing and a first activation position relative to the housing. The user activation device further includes a switch that is configured to control the supply of the electrosurgical energy to the electrosurgical electrode responsive to the collar moving from the home position to the first activation position.

At block 812, the process includes moving the collar from the home position to the first activation position. Responsive to moving the collar from the home position to the first activation position at block 812, the process 800 includes supplying the electrosurgical energy from the electrosurgical generator to the electrosurgical electrode at block 814.

FIGS. 9-16 depict additional aspects of the process 800 according to further examples. As shown in FIG. 9, the process 800 can include (i) after supplying the electrosurgical energy at block 814, moving the collar from the first activation position to the home position at block 816, and (ii) responsive to moving the collar from the first activation position to the home position at block 816, ceasing the supply of the electrosurgical energy to the electrosurgical electrode at block 818.

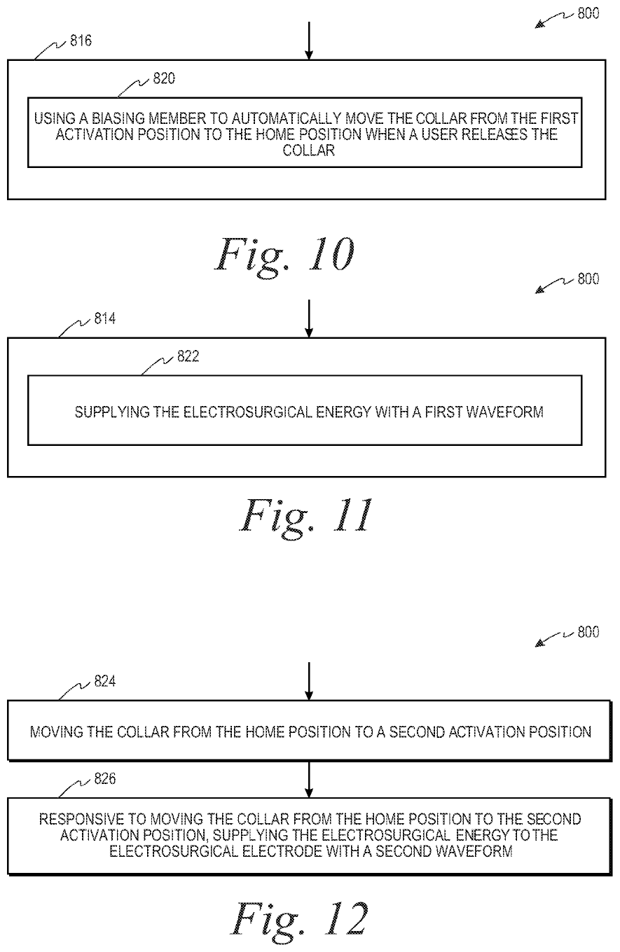

As shown in FIG. 10, moving the collar from the first activation position to the home position at block 816 can include using a biasing member to automatically move the collar from the first activation position to the home position when a user releases the collar at block 820.

As shown in FIG. 11, supplying the electrosurgical energy from the electrosurgical generator to the electrosurgical electrode responsive to moving the collar from the home position to the first activation position at block 814 can include supplying the electrosurgical energy with a first waveform at block 822.

As shown in FIG. 12, the process 800 can further include moving the collar from the home position to a second activation position at block 824. The home position is between the first activation position and the second activation position. Responsive to moving the collar from the home position to the second activation position at block 824, the process 800 can include supplying the electrosurgical energy to the electrosurgical electrode with a second waveform at block 826. The first waveform is different from the second waveform.

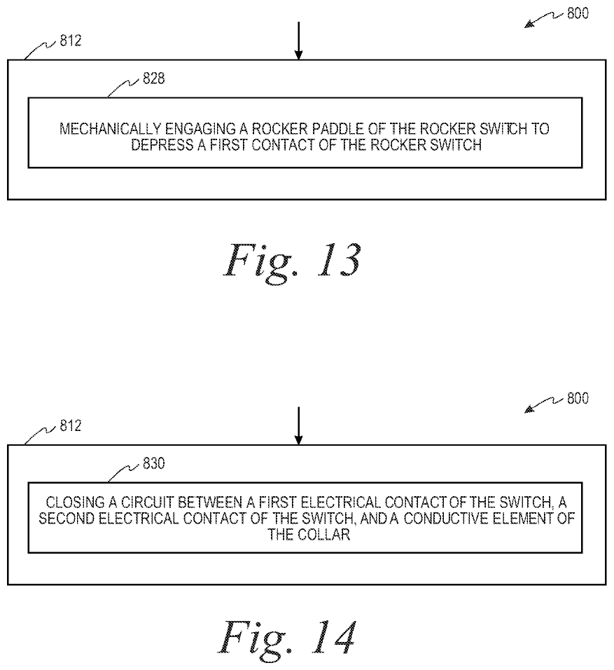

As shown in FIG. 13, in an example in which the switch includes a rocker switch, moving the collar from the home position to the first activation position at block 812 can include mechanically engaging a rocker paddle of the rocker switch to depress a first contact of the rocker switch at block 828.

As shown in FIG. 14, moving the collar from the home position to the first activation position at block 812 can include closing a circuit between a first electrical contact of the switch, a second electrical contact of the switch, and a conductive element of the collar at block 830.

As shown in FIG. 15, the process 800 can further include (i) sensing, by a Hall Effect sensor, that the collar is in the home position at block 832, (ii) responsive to sensing that the collar is in the home position at block 832, preventing supply of the electrosurgical energy to the electrosurgical electrode at block 834, and (iii) sensing, by the Hall Effect sensor, that the collar is in the first activation position at block 836. Additionally, as shown in FIG. 15, supplying the electrosurgical energy from the electrosurgical generator to the electrosurgical electrode at block 814 can be responsive to sensing that the collar is in the first activation position at block 832.

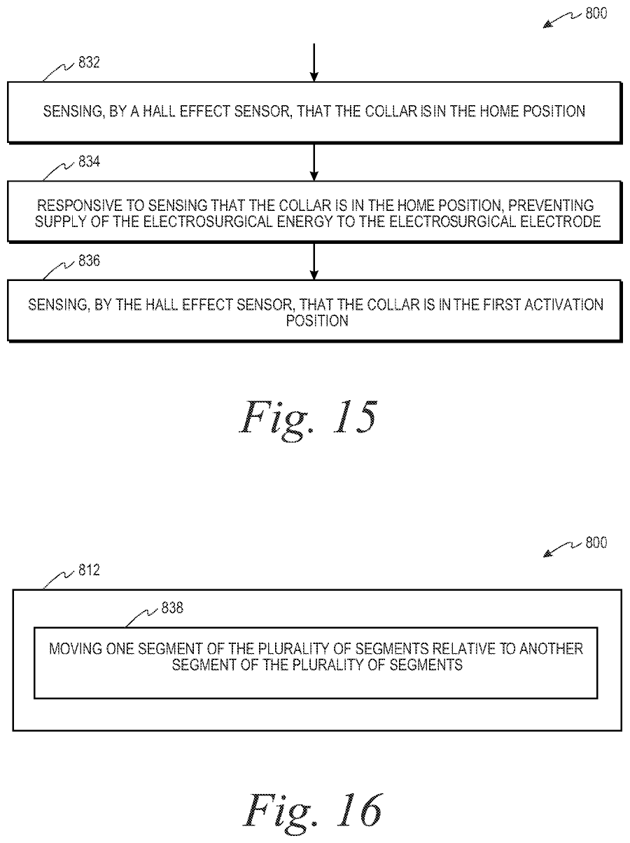

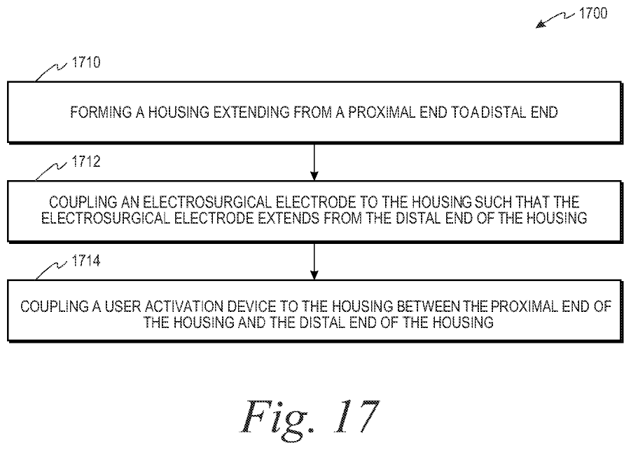

As shown in FIG. 16, in an example in which the collar includes a plurality of segments that are independently movable relative to each other and the housing, moving the collar from the home position to the first activation position at block 812 can include moving one segment of the plurality of segments relative to another segment of the plurality of segments at block 838.

FIG. 17 depicts a flowchart for a method 1700 for manufacturing an electrosurgical instrument, according to an example. At block 1710, the process 1700 can include forming a housing extending from a proximal end to a distal end. At block 1712, the process 1700 can include coupling an electrosurgical electrode to the housing such that the electrosurgical electrode extends from the distal end of the housing. At block 1714, the process 1700 can include coupling a user activation device to the housing between the proximal end of the housing and the distal end of the housing.

The user activation device is operable to control a supply of electrosurgical energy to the electrosurgical electrode. The user activation device includes a collar that extends around at least a half of a circumference of the housing. The collar is movable between a home position relative to the housing and a first activation position relative to the housing. The user activation device also includes a switch that is configured to control the supply of the electrosurgical energy to the electrosurgical electrode responsive to the collar moving from the home position to the first activation position.

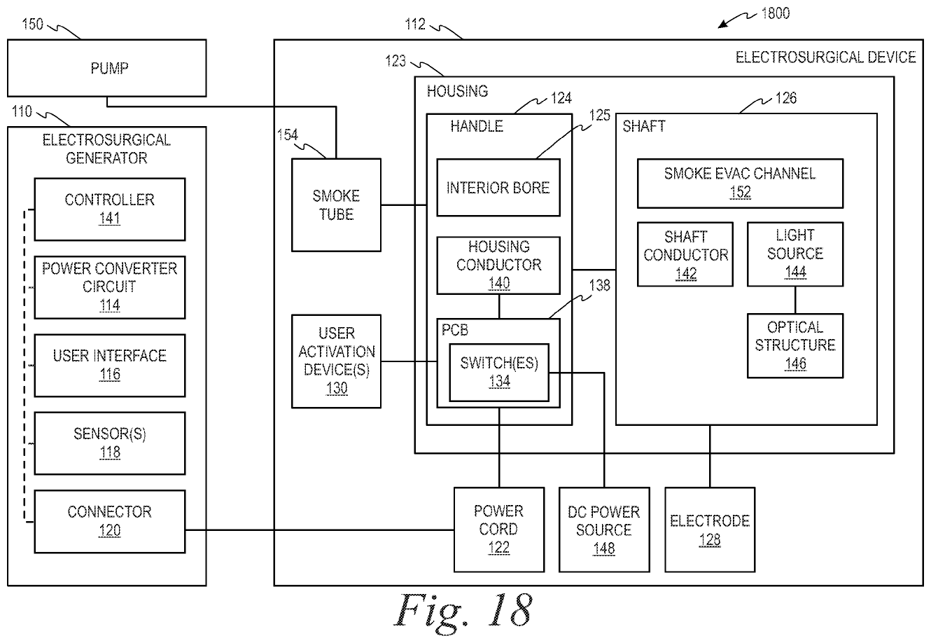

Referring to now to FIG. 18, an electrosurgical system 1800 is shown according to another example. As shown in FIG. 18, the electrosurgical system 1800 includes the electrosurgical generator 110 and the electrosurgical device 112. As described above, the electrosurgical generator 110 can generate electrosurgical energy that is suitable for performing electrosurgery on a patient. For instance, the electrosurgical generator 110 can include the power converter circuit 114 described above, which can convert a grid power to electrosurgical energy such as, for example, a radio frequency (RF) output power. Additionally, as described above, the electrosurgical generator 110 can include the user interface 116, the one or more generator sensors 118, the connector 120, and/or the controller 141.

As described above with respect to FIG. 1, the housing 123 of the electrosurgical instrument 112 can include the handle 124, the shaft 126, and the electrosurgical electrode 128.

In some implementations, the shaft 126 can be coupled to the handle 124 in a fixed and non-moveable manner. This may simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical device 112). This can provide for adjusting a length of the electrosurgical device 112, which can facilitate performing electrosurgery at a plurality of different depths within tissue (e.g., due to different anatomical shapes and/or sizes of patients) and/or at a plurality of different angles.

In some implementations, the electrosurgical electrode 128 can additionally or alternatively be rotatable about an axis of rotation that is parallel to the longitudinal axis of the electrosurgical device 112. In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124 and at least one additional component in the inner cavity defined by the shaft 126. Rotating the electrosurgical electrode 128 relative to the handle 124 can facilitate adjusting an angle of the electrosurgical electrode 128 relative to one or more user activation device(s) 130 of the electrosurgical device 112. In this arrangement, a user can comfortably grip the handle 124 in a position in which their fingers can comfortably operate the user activation device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

Although it can be beneficial to provide for rotation of the electrosurgical electrode 128 relative to the handle 124 and/or the shaft 126, the electrosurgical electrode 128 can be rotationally fixed relative to the handle 124 and the shaft 126 in some implementations. This may, for example, help to simplify manufacturing and reduce a cost of manufacture by, for instance, simplifying electrical connections that may otherwise need to account for movement of the shaft 126 and the handle 124 relative to each other (e.g., by omitting slip ring electrical contacts and/or sliding electrical contacts). As described in further detail below, the user activation device(s) 130 of the electrosurgical device 112 described herein can help to reduce or obviate a need to rotate the electrosurgical electrode 128 relative to the handle 124.

As described above, the user activation device(s) 130 can select between the modes of operation of the electrosurgical device 112 and/or the electrosurgical generator 110. For instance, in one implementation, the user activation device(s) 130 can be configured to select between a cutting mode of operation and a coagulation mode of operation. Responsive to actuation of the user activation device(s) 130 of the electrosurgical device 112, the electrosurgical device 112 can (i) receive the electrosurgical energy with a level of power and/or a waveform corresponding to the mode of operation selected via the user activation device(s) 130 and (ii) supply the electrosurgical energy to the electrosurgical electrode 128.

In FIG. 18, the electrosurgical device 112 includes a plurality of electrical components that facilitate supplying the electrosurgical energy, which the electrosurgical device 112 receives from the electrosurgical generator 110, to the electrosurgical electrode 128. For example, the electrosurgical device 112 can include at least one electrical component selected from a group of electrical components including: the printed circuit board 138 (e.g., a flexible printed circuit board), the housing conductor 140, and/or the shaft conductor 142 that can provide a circuit for conducting the electrosurgical energy from the power cord 122 to the electrosurgical electrode 128. One or more of the electrical components can be positioned in the interior bore defined by the handle 124 and/or in the inner cavity defined by the shaft 126.

Within examples, the user activation device(s) 130 can include one or more buttons on an exterior surface of the handle 124. Each button of the user activation device(s) 130 can be operable to actuate a respective one of a plurality of switches 134 coupled to the printed circuit board 138. In general, the switches 134 and/or the printed circuit board 138 are operable to control a supply of the electrosurgical energy from the electrosurgical generator 110 to the electrosurgical electrode 128. For instance, in one implementation, when each button is operated (e.g., depressed), the respective switch 138 associated with the button can be actuated to cause the printed circuit board 138 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 138 associated with the button can close the switch 138 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 138 can be omitted.

In both example implementations, the electrosurgical energy supplied by the electrosurgical generator 110 can be supplied from (i) the power cord 122, the printed circuit board 138, and/or the switches 134 to (ii) the electrosurgical electrode 128 by the housing conductor 140 and the shaft conductor 142. As such, as shown in FIG. 18, the printed circuit board 138 can be coupled to the power cord 122, the housing conductor 140 can be coupled to the printed circuit board 138 and the shaft conductor 142, and the shaft conductor 142 can be coupled to the electrosurgical electrode 128. In this arrangement, the housing conductor 140 can conduct the electrosurgical energy (supplied to the housing conductor 140 via the printed circuit board 138) to the shaft conductor 142, and the shaft conductor 142 can conduct the electrosurgical energy to the electrosurgical electrode 128.

In general, the housing conductor 140 and the shaft conductor 142 can each include one or more electrically conductive elements that provide an electrically conductive bus for supplying the electrosurgical energy to the electrosurgical electrode 128. More particularly, the housing conductor 140 can include one or more electrically conductive elements of the handle 124 that can supply the electrosurgical energy to the shaft conductor 142, and the shaft conductor 142 can include one or more electrically conductive elements of the shaft 126 that can supply the electrical energy from the housing conductor 140 to the electrosurgical electrode 128. As described in further detail below, the housing conductor 140 can engage the shaft conductor 142 to maintain an electrical coupling between the housing conductor 140, the shaft conductor 142, and the electrosurgical electrode 128 while (i) the shaft 126 and/or the electrosurgical electrode 128 telescopically moves relative to the handle 124, and/or (ii) the electrosurgical electrode 128 rotates relative to the handle 124. As described above, the electrosurgical electrode 128 can apply the electrosurgical energy to a target tissue to perform an electrosurgical operation (e.g., cutting, coagulating, ablating, and/or sealing the target tissue). The electrosurgical electrode 128 can be configured as described above (e.g., with the shapes, the materials, etc. described above).

As shown in FIG. 18, in some implementations, the electrosurgical device 112 can additionally include the one or more light sources 144 that are configured to emit light. In examples that include the light source(s) 144, the light source(s) 144 can generate light that can be emitted by the electrosurgical device 112 to illuminate an area of interest (e.g., a target tissue at the surgical site). In some examples that include the light source(s) 144, the user activation device 130 can be operable to cause the light source(s) 144 to generate light that can be emitted by the electrosurgical instrument 112 to illuminate an area of interest (e.g., a target tissue at the surgical site). In some implementations, the light source(s) 144 can be located at a distal end of the housing 123 and/or a distal end of the shaft 126 to directly provide light in a distal direction and illuminate a surgical distal of the electrosurgical electrode 128.

In other implementations, as shown in FIG. 18, the light source(s) 144 can be optically coupled to the optical structure 146, which is configured to receive the light emitted by the light source(s) 144 and transmit the light in a distal direction toward a surgical site to illuminate the surgical site while performing electrosurgery using the electrosurgical electrode 128. Although arranging the light source(s) 144 to directly illuminate a surgical field can help, for instance, to reduce a cost of manufacture, transmitting the light using the optical structure 146 can help to improve a quality of light transmitted from the electrosurgical device 112 (e.g., by providing light with improved uniformity and/or reduced heat generation).

As examples, in implementations that include the optical structure 146, the optical structure 146 can include at least one optical structure selected from among a group consisting of an optical lens, a non-fiber optic optical waveguide, and an optical fiber. When the optical structure 146 includes the optical lens (e.g., a parabolic reflector lens, an aspheric lens, and/or a Fresnel lens), the optical structure 146 can help to direct the light emitted by the light source 144 in the distal direction and thereby improve a quality of the light illuminating the surgical site. The optical structure 146 can additionally or alternatively include the non-fiber optic optical waveguide and/or the optical fiber to transmit the light over relatively large distances in the shaft 126. For instance, the optical waveguide can transmit the light in the distal direction via total internal reflection. In such implementations, the optical waveguide can include a cladding and/or an air gap on an exterior surface of the optical waveguide to help facilitate total internal reflection. In some implementations, the non-fiber optic optical waveguide can be formed as a single, monolithic structure.

In some examples, the optical structure 146 can additionally or alternatively include other light shaping optical elements such as, for instance, a plurality of facets, one or more prisms, and/or one or more optical gratings. Although the optical structure 146 can help to improve a quality of the light directed to the surgical site, the electrosurgical device 112 can omit the optical structure 146 and instead emit the light from the light source 144 directly to the surgical field without transmitting the light through the optical structure 146 in other examples.

In FIG. 18, the light source 144 can be coupled to the shaft 126. As such, the light source 144 can also move telescopically with the shaft 126 relative to the handle 124. However, in other examples, the light source 144 can be in the interior bore of the handle 124 and/or coupled to an exterior surface of the handle 124. As examples, the light source 144 can include one or more light emitting diodes (LEDs), organic light emitting diodes (OLEDs), optical fibers, non-fiber optic waveguides, and/or lenses. Additionally, for example, the light source 144 can include a LED printed circuit board having one or more light sources (e.g., LEDs).

The optical structure 146 can be at a distal end of the shaft 126. In some examples, the optical structure 146 can circumferentially surround the electrosurgical electrode 128 to emit the light distally around all sides of the electrosurgical electrode 128. This can help to mitigate shadows and provide greater uniformity of illumination in all rotational alignments of the shaft 126 relative to the housing 123 and/or the electrosurgical device 112 relative to the target tissue. However, in other examples, the optical structure 146 can extend partially but not fully around the electrosurgical electrode 128.

In implementations that include the light source 144, the user activation device(s) 130, the printed circuit board 138, the switches 134, the housing conductor 140, and/or the shaft conductor 142 can additionally supply an electrical power from a direct current (DC) power source 148 to the light source 144. In one example, the DC power source 148 can include a battery disposed in the handle 124, the plug of the power cord 122, and/or a battery receptacle located along the power cord 122 between the handle 124 and the plug. Although the electrosurgical device 112 includes the DC power source 148 in FIG. 18, the DC power source 148 can be separate and distinct from the electrosurgical device 112 in other examples. For instance, in another example, the electrosurgical generator 110 can include the DC power source 148.

Additionally, in implementations that include the light source 144, the user activation device(s) 130 can be operable to cause the light source 144 to emit the light. In one example, the user activation device(s) 130 can include a button that independently controls the light source 144 separate from the button(s) that control the electrosurgical operational modes of the electrosurgical device 112. In another example, the user activation device(s) 130 and the printed circuit board 138 can be configured such that operation of the button(s) that control the electrosurgical operational mode simultaneously control operation of the light source 144 (e.g., the light source 144 can be automatically actuated to emit light when a button is operated to apply the electrosurgical energy at the electrosurgical electrode 128).

As shown in FIG. 18, responsive to operation of the user activation device(s) 130 to actuate the light source 144, the DC power source 148 can supply the electrical power (e.g., a DC voltage) to the light source 144 via the printed circuit board 138, the housing conductor 140, and/or the shaft conductor 142. In this implementation, one or more of the conductive elements of the housing conductor 140 can be configured to supply the electrical power from the DC power source 148 to the light source 144 and/or return the electrical power from the light source 144 to the DC power source 148. Accordingly, the housing conductor 140 can additionally or alternatively assist in providing electrical communication between the DC power source 148 and the light source 144 as the shaft 126 and the light source 144 telescopically move relative to the handle 124.

Although the user activation device(s) 130 on the handle 124 can be operated to control the operation of the light source 144 in the examples described above, the light source 144 can be additionally or alternatively operated by one or more user activation device(s) on the electrosurgical generator 110 (e.g., via the user interface 116) and/or on the plug of the power cord 122.

In some examples, the electrosurgical device 112 can additionally or alternatively include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include the suction pump 150 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 150 and the electrosurgical generator 110 can be provided as separate devices or integrated in a single device (e.g., in a common housing).

As shown in FIG. 18, the shaft 126 can include the smoke evacuation channel 152 in the inner cavity of the shaft 126. The smoke evacuation channel 152 can also include a smoke inlet that can extend circumferentially around a center axis of a distal portion of the electrosurgical electrode 128. In this arrangement, the smoke inlet of the smoke evacuation channel can help to receive surgical smoke into the smoke evacuation channel 152 in all rotational alignments of the electrosurgical electrode 128 relative to the handle 124 and/or the electrosurgical device 112 relative to the target tissue. However, in another example, the smoke evacuation channel 152 can include one or more smoke inlets that do not extend circumferentially around the electrosurgical electrode 128.

In an example, the smoke evacuation channel 152 can include an outer tube that is separated from the optical lens assembly 142 by an air gap. For instance, the shaft 126 can include a plurality of standoffs that extend between the optical lens assembly 142 and the outer tube of the smoke evacuation channel 152 to provide the air gap between the outer tube and the optical lens assembly 142. In one implementation, the optical lens assembly 142 can include the standoffs such that the optical lens assembly 142 and the standoffs are formed as a single, monolithic structure. In another implementation, the standoffs can be formed as a single, monolithic structure with the outer tube of the smoke evacuation channel 152. In another implementation, the standoffs can be separate from the outer tube of the smoke evacuation channel 152 and the optical lens assembly 142.

As described above, the electrosurgical electrode 128 shown in FIG. 18 can be rotatable about an axis of rotation that is parallel to the longitudinal axis of the electrosurgical device 112. Rotating the electrosurgical electrode 128 relative to the handle 124 can facilitate adjusting an angle of the electrosurgical electrode 128 relative to one or more user activation device(s) 130 of the electrosurgical device 112. In this arrangement, a user can comfortably grip the handle 124 in a position in which their fingers can comfortably operate the user activation device(s) 130 while the electrosurgical electrode 128 is set at a rotational position selected from among a plurality of rotational positions relative to the handle 124 based on, for example, a location, a size, and/or a shape of a surgical site in which the user is operating. In particular, the user activation device(s) 130 can include one or more buttons on an exterior surface of the handle 124, where the one or more buttons that are operable to select between a cutting mode of operation and a coagulation mode of operation.

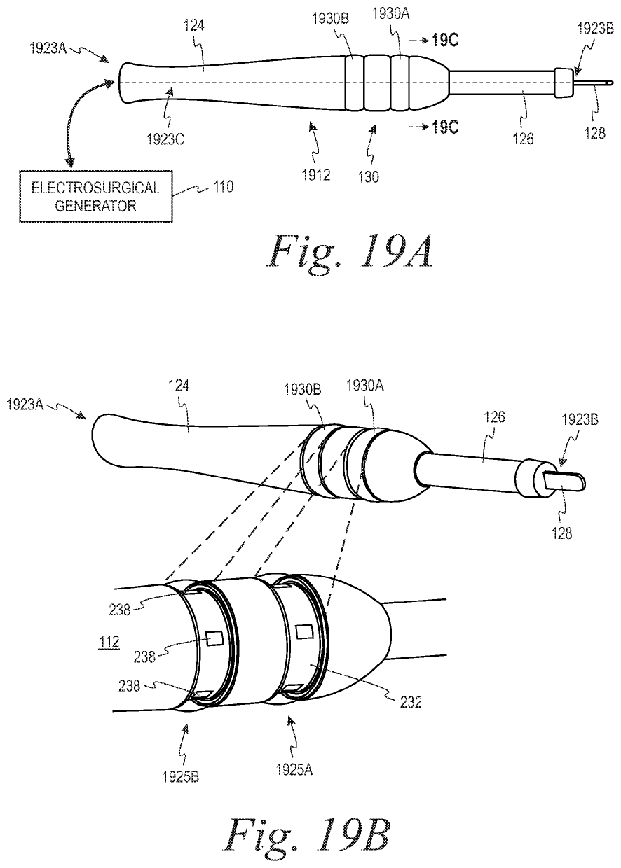

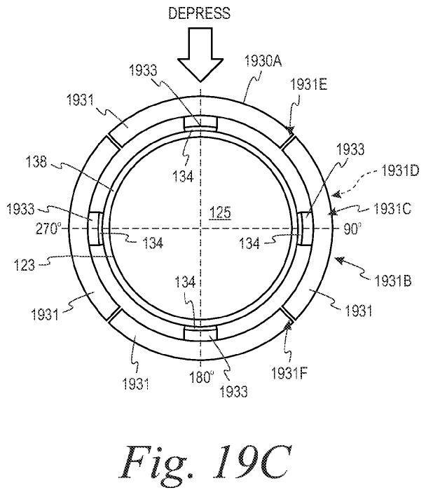

In alternative examples, electrosurgical devices can be configured to reduce the need to rotate an electrode relative to the handle to allow comfortable operation of user activation device(s). For instance, referring to FIG. 19A, an electrosurgical device 1912 includes the housing 123 extending along a longitudinal axis 1923C between a proximal end 1923A and a distal end 1923B. The housing 123 includes the handle 124 defining the proximal end 1923A and the shaft 126 defining the distal end 1923B. The electrosurgical device 1912 includes the electrosurgical electrode 128 that is coupled to the shaft 126 so that the electrosurgical electrode 128 extends from the distal end 1923B of the housing 123. The electrosurgical device 1912 includes user activation devices 130. In particular, the user activation devices 130 include a first user activation device 1930A that is operable to control a supply of electrosurgical energy to the electrosurgical electrode 228. The first user activation device 1930A extends a sufficient length along a circumference of the housing 123 so that a user's fingers can reach and operate the first user activation device 1930A while the electrosurgical electrode 128 remains in a preferred position and orientation for cutting, coagulation, etc., at a surgical site. The length of the first user activation device 1930A around the housing 123 accommodates preferred positioning and orientation of the electrosurgical electrode 128 without needing relative rotation between the electrosurgical electrode 128 and the handle 124 (as described above with reference to FIG. 18).

In some implementations, the first user activation device 1930A extends around at least half of a circumference of the housing 123. As shown in FIG. 19B, for example, the first user activation device 1930A defines a ring that extends around an entirety of the circumference of the housing 123. The first user activation device 1930A can be disposed in a channel 1925A in the housing 123, where the channel 1925A is recessed relative to an exterior surface of the housing 123.

FIG. 19C illustrates a cross-sectional view of the electrosurgical device 1912 at the first user activation device 1930A. The first user activation device 1930A includes a plurality of segments 1931, where each segment 1931 extends around a respective portion of the circumference of the housing 123. As shown in FIG. 19C, four segments 1931 are positioned respectively at 0°, 90°, 180°, and 2700 around the circumference of the housing 123 to define the ring in the channel 1925A. The first user activation device 1930A in other examples, however, can have a different number of segments 1931 (e.g., two, three, five, or more segments 1931). Furthermore, although the segments 1931 in FIG. 19C may define a circular arrangement around a circular profile for the housing 123, the housing 123 in other examples can have a non-circular (e.g., elliptical) profile and the segments 1931 can define a corresponding non-circular shape around the housing 123.

As shown in FIG. 19C, each segment 1931 includes an inner surface 1931A that faces the housing 123, an outer surface 1931B that faces away from the housing 123 and opposes the inner surface 1931A, a distal surface 1931C, a proximal surface 1931D, a first lateral surface 1931E, and a second lateral surface 1931F. As shown in FIG. 19C, the inner surface 1931A of each segment 1931 has a common radius of curvature, and the plurality of segments 1931 have the same shape and the same size as each other. In other examples, however, the segments 1931 may have different shapes and sizes. For instance, if the housing 123 has an elliptical profile, the first user activation device 1930A may include two pairs of symmetrically opposing segments 1931, where the segments 1931 in one pair are different in shape and size from the segments 1931 in the other pair.

In some implementations, only one segment 1931 of the plurality of segments 1931 is operable at a time, i.e., when one segment 1931 is depressed, the other segments 1931 cannot be concurrently depressed. For example, the first user activation device 1930A can be configured such that, when one segment 1931 (e.g., at 0°) is depressed towards the housing 123, the one segment 1931 engages adjacent segments 1931 (e.g., at 900 and 270°) and stops the adjacent segments 1931 from being depressed towards the housing 123.

In an implementation, the electrosurgical device 1912 can include the plurality of switches 134, where the first user activation device 1930A engages the plurality of switches 134. Each switch 134 is disposed between a respective one of the plurality of segments 1931 and the housing 123 and is configured to be actuated to control the supply of electrosurgical energy to the electrosurgical electrode 128. For example, the switches 134 can be positioned at 0°, 90°, 180°, and 270° corresponding to positions of the segments 1931. A switch 134 can be individually actuated when the respective segment 1931 is depressed radially inward into the switch 134. In an implementation, depressing the segment 1931 and thereby actuating the respective switch 134 associated with the segment 1931 can close the switch 134 to complete a circuit to a electrosurgical generator 110 coupled to the electrosurgical device 1912 (as described above). The completed circuit causes the electrosurgical generator 110 to supply the electrosurgical energy to the electrosurgical electrode 128 with a level of power and/or a waveform corresponding to a mode of operation associated with the first user activation device 1930A.

In some implementations, the electrosurgical device 1912 includes one or more flexible printed circuit boards 138, where the plurality of switches 134 are configured to cause the one or more flexible printed circuit boards 138 to control the supply of electrosurgical energy to the electrosurgical electrode 128. The one or more flexible printed circuit boards 138 are disposed between the switches 134 and the housing 123. In an example, the switches 134 engage a common flexible printed circuit board 138. In another example, each switch 134 engages a respective one of a plurality of flexible printed circuit boards 138 (e.g., positioned at 0°, 90°, 180°, and 270°). When actuated, each switch 134 causes a flexible printed circuit board 134 to transmit a signal to an electrosurgical generator 110. The electrosurgical generator 110 then responsively supplies electrosurgical energy to the electrosurgical electrode 128 with a level of power and/or a waveform corresponding to a mode of operation associated with the first user activation device 1930A.

As shown in FIG. 19C, the combination of a segment 1931 and a switch 134 at each of multiple positions around the longitudinal axis 1923C (e.g., at 0°, 90°, 180°, and 270°) allows a user to depress and operate the first user activation device 1930A from multiple angles or approaches. As such, the first user activation device 1930A is considered a multi-axial user activation device. This multi-axial user activation device allows the user to hold the electrosurgical device 1912 in a manner that accommodates a preferred position and orientation for the electrosurgical electrode 128 while comfortably operating the first user activation device 1930A according to the mode of operation. Advantageously, this reduces the need to stop and restart a surgical procedure to allow the user to rotate the electrosurgical electrode 128 relative to the handle 124 to maintain the preferred position and orientation for the electrosurgical electrode 128. Furthermore, this multi-axial user activation device provides ambidextrous functionality (i.e., accommodates operation by right or left hand).

In an example, each segment 1931 moves or deforms appreciably radially inward into the corresponding switch 134 when depressed and moves or reforms appreciably radially outward when no longer depressed. With this inward and outward radial movement or deformation, a user can receive sufficient tactile feedback when operating the first user activation device 1930A. To provide such feedback, the segment 1931 can be formed from a compressible material that is reversibly deformed when the segment 1931 is depressed. Examples of a compressible material can include, but are not limited to, any elastomer (e.g., silicone), nitrile butadiene rubber (NBR), ethylene propylene diene monomer (EPDM) rubber, or the like. Alternatively, the segment 1931 can be formed from a non-compressible material, including, but not limited to, acrylonitrile butadiene styrene (ABS), high impact polystyrene (HIPS), poly(methyl methacrylate) (PMMA), or any hard engineering plastic. A bias element, such as a spring, can be disposed between each segment 1931 and the housing 123, where the bias element acts to bias the segment 1931 radially outward when not depressed.

Each segment 1931 can be movably coupled to the housing 123. Alternatively or additionally, each segment 1931 can be movably coupled to adjacent segments 1931. Alternatively or additionally, a sheath formed from a flexible material or similar device can fit over the segments 1931 to hold the segments 1931 in place in the channel 1925A.

In further implementations, the first user activation device 1930A includes at least one sensor 1933 that is configured to sense an extent to which each segment 1931 is depressed towards the housing 123. The at least one sensor 1933 can be one or more pressure sensors, one or more optical sensors, one or more capacitive sensors, one or more inductive sensors, one or more infrared sensors, one or more piezoelectric sensors, one or more ultrasonic sensors, one or more resistive sensors, and/or one or more image sensors (e.g., camera). In one implementation, as shown in FIG. 19C, each segment 1931 is coupled to a respective sensor 1933 to sense the extent to which the segment 1931 is depressed. The switch 134 can be actuated when the sensor 1933 detects sufficient contact or movement when the respective segment 1931 is depressed. For example, each switch 134 can be actuated when the sensor 1933 detects that the respective segment 1931 has been depressed to apply a pressure that exceeds a threshold pressure over a threshold period of time. As such, the sensor 1933 reduces the chance of accidental actuation with the first user activation device 1930A. In general, the sensors 1933 can detect operation of the first user activation device 1930A irrespective of what part of the first user activation device 1930A is depressed.

As shown in FIGS. 19B-C, the electrosurgical device 1912 can also include a second user activation device 1930B that is offset along the longitudinal axis 1923C of the housing 123 relative to the first user activation device 1930A, where the longitudinal axis 1923C extends between the proximal end 1923A and the distal end 1923B of the housing 123. According to one implementation, the first user activation device 1930A is operable to supply the electrosurgical energy with a first waveform that is configured for cutting tissue with the electrosurgical electrode 128, while the second user activation device 1930B is operable to supply electrosurgical energy with a second waveform that is configured for coagulating tissue with the electrosurgical electrode 128, where the first waveform is different than the second waveform.

The second user activation device 1930B can include the features of the first user activation device 1930A described herein. In particular, similar to the first user activation device 1930A, the second user activation device 1930B extends a sufficient length along a circumference of the housing 123 so that a user's fingers can reach and operate the second user activation device 1930B while the electrosurgical electrode 128 remains in a preferred position and orientation at a surgical site. The length of the second user activation device 1930B accommodates preferred positioning and orientation of the electrosurgical electrode 128 without needing relative rotation between the electrosurgical electrode 128 and the handle 124 (as described above with reference to FIG. 18). In general, the second user activation device 1930B can provide another a multi-axial user activation device for additional operation of the electrosurgical electrode 128.

In some implementations, the second user activation device 1930B extends around at least half of the circumference of the housing 123. As shown in FIG. 19B, for example, the second user activation device 1930B defines a second ring that extends around an entirety of the circumference of the housing 123. The second user activation device 1930B can be disposed in a channel 1925B in the housing 123, where the channel 1925B is recessed relative to an exterior surface of the housing 123. In other implementations, the electrosurgical device 1912 can include additional user activation devices with similar features.

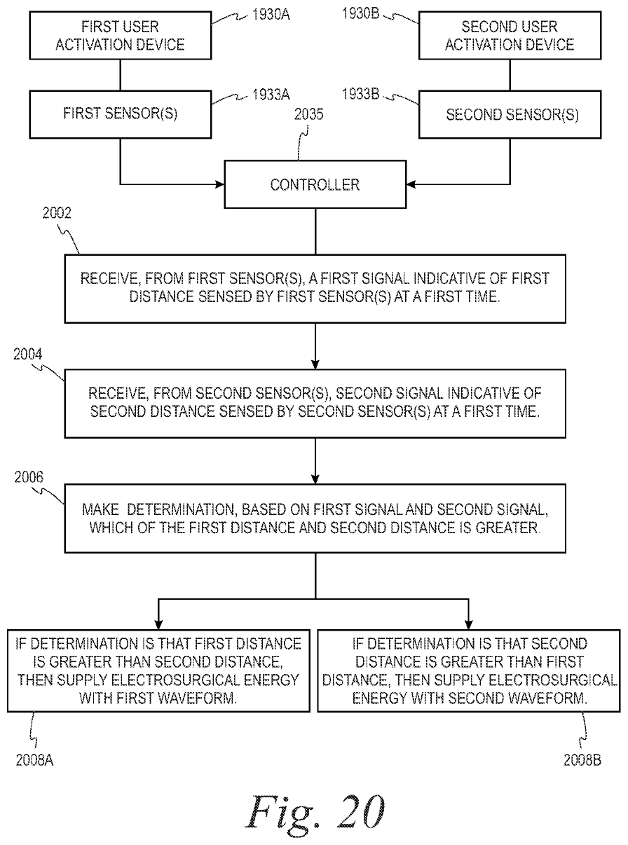

Referring to an example system illustrated in FIG. 20, the first user activation device 1930A includes at least one first sensor 1933A that is configured to sense a first distance by which the first user activation device 1930A is depressed towards the housing 123, and the second user activation device 1930B includes at least one second sensor 1933B that is configured to sense a second distance by which the second user activation device 1930B is depressed towards the housing 123. In some cases, the first user activation device 1930A and the second user activation device 1930B might be depressed at the same time. In such cases, the electrosurgical device 1912 can determine whether to respond to the first user activation device 1930A or the second user activation device 1930B. In particular, the electrosurgical device 1912 includes a controller 2035 that is in communication with the at least one first sensor 1933A and the at least one second sensor 1933B. In an act 2002, the controller 2035 receives, from the at least one first sensor 1933A, a first signal indicative of the first distance sensed by the at least one first sensor 1933A at a first time. In an act 2004, the controller 2035 receives, from the at least one second sensor 1933B, a second signal indicative of the second distance sensed by the at least one second sensor 1933A at the first time. In an act 2006, the controller 2035 makes a determination, based on the first signal and the second signal, which of the first distance and the second distance is greater. If the determination is that the first distance is greater than the second distance, then controller 2035 in act 2008A supplies the electrosurgical energy the electrosurgical electrode 128 with the first waveform, which causes the electrosurgical electrode 128 to cut tissue. On the other hand, if the determination is that the second distance is greater than the first distance, then controller 2035 in act 2008B supplies the electrosurgical energy the electrosurgical electrode 128 with the second waveform, which causes the electrosurgical electrode 128 to coagulate tissue. Accordingly, in this example, the electrosurgical device 1912 can determine what mode of operation the user intends to actuate.

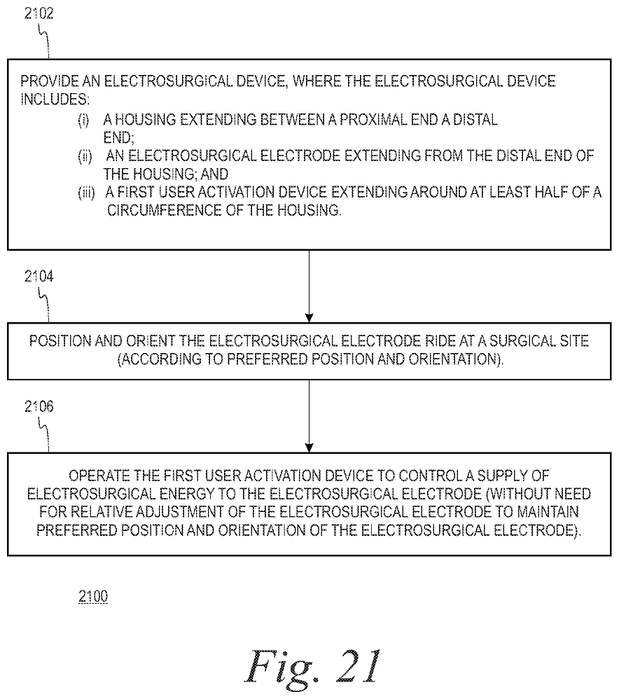

FIG. 21 depicts a flowchart for a method 2100 for operating an electrosurgical device, according to an example. In the method 2100, act 2102 includes providing an electrosurgical device, where the electrosurgical device includes: (i) a housing extending between a proximal end and a distal end; (ii) an electrosurgical electrode extending from the distal end of the housing; and (iii) a first user activation device extending around at least half of a circumference of the housing. Act 2104 includes positioning and orienting the electrosurgical electrode at a surgical site. Act 2106 includes operating the first user activation device to control a supply of electrosurgical energy to the electrosurgical electrode. Advantageously, the user activation device can be operated in act 2106 without needing to adjust the electrosurgical electrode to maintain a preferred position and orientation for the electrosurgical electrode established in act 404.

Although the features above may be described in relation to electrosurgery, it is understood that such features can be more generally implemented in any medical instrument for any medical procedure. In particular, one or more multi-axial user activation devices may be employed to allow comfortable hand operation of a medical instrument while maintaining a preferred position and orientation for the medical instrument.

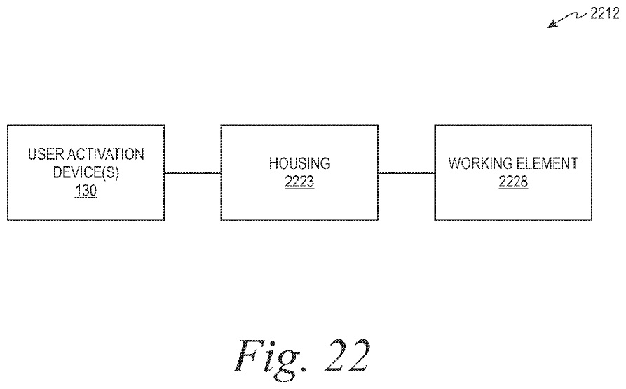

As an example, FIG. 22 depicts a surgical instrument 2212 that is operable to perform a surgical task during a surgical procedure. As examples, the surgical instrument 2212 can include at least one instrument selected from among a group consisting of a drill, a bone cutter, an aspiration tool, an irrigation tool, a shaver, a microscope, a camera (e.g., an endoscope), a surgical retractor, an electrosurgical instrument, and an illumination device. Within examples, the surgical instrument 2212 can include a working element 228 that is operable to perform the at least one surgical task selected from a group consisting of a drilling operation, a cutting operation, a shaving operation, a tissue retraction operation, a suctioning operation, an irrigation operation, a probing operation, a clamping operation, a coagulation operation, a heating operation, a cooling operation, an ablation operation, an electrical stimulation operation, an image capture operation, a sawing operation, and a grinding operation. For instance, the working element 2228 can include a drill bit, an electrosurgical electrode (e.g., the electrode 128 described above), an ablation end effector (e.g., a cryoablation balloon, an electrode, a laser light emitter, and/or a heating element), a fluid valve, a vacuum source, and a cutting blade.

As shown in FIG. 22, the surgical instrument 2212 can include a housing 2223 and the working element 2228 can be coupled to the housing 2223. The housing 2223 can be similar to the housing 123 described above. For instance, in some examples, the housing 2223 can include the handle 124 and the shaft 126. More generally, the housing 123 can be gripped, manipulated, and moved by the surgeon during the surgical procedure.

Additionally, as shown in FIG. 22, the surgical instrument 2212 includes one or more of the activation devices 130 described above with respect to FIGS. 1-21. In this arrangement, the user activation device(s) 130 that can be actuated to operate the working element 2228 of the surgical instrument 2212 from multiple angles or approaches. As described above, this reduces the need to stop and restart a surgical procedure to allow the user to adjust the working element 2228 to maintain the preferred position and orientation for the working element 2228. Furthermore, this user activation device provides ambidextrous functionality (i.e., accommodates operation by right or left hand).

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may provide different advantages as compared to other advantageous embodiments. The implementation or implementations selected are chosen and described in order to best explain the principles of the embodiments, the practical application, and to enable others of ordinary skill in the art to understand the disclosure for various embodiments with various modifications as are suited to the particular use contemplated.

What is claimed is:

1. An electrosurgical instrument, comprising:
a housing extending from a proximal end to a distal end;
an electrosurgical electrode extending from the distal end of the housing; and
a user activation device disposed between the proximal end of the housing and the distal end of the housing, wherein the user activation device is operable to control a supply of electrosurgical energy to the electrosurgical electrode,
wherein the user activation device comprises:
a collar that extends around at least a half of a circumference of the housing, wherein the collar is movable between a home position relative to the housing and a first activation position relative to the housing, and
a switch that is configured to control the supply of electrosurgical energy to the electrosurgical electrode responsive to the collar moving from the home position to the first activation position.

2. The electrosurgical instrument of claim 1, wherein the collar extends around an entirety of the circumference of the housing.

3. The electrosurgical instrument of any one of claims 1-2, wherein the collar is further movable between the home position and a second activation position relative to the housing, wherein the home position is between the first activation position and the second activation position, and
wherein, responsive to the collar moving from the home position to the first activation position, the switch is configured to cause the electrosurgical energy to be supplied to the electrosurgical electrode with a first waveform,
wherein, responsive to the collar moving from the home position to the second activation position, the switch is configured to cause the electrosurgical energy to be supplied to the electrosurgical electrode with a second waveform,
wherein the first waveform is different from the second waveform, and
wherein, wherein when the collar is in the home position, the switch is configured to prevent the electrosurgical energy from being supplied to the electrosurgical electrode.

4. The electrosurgical instrument of claim 3, wherein the switch comprises a rocker switch that is mechanically actuatable between a first position, a second position, and a third position, and
wherein the collar is configured to mechanically engage the switch such that (i) the switch is in the first position when the collar is in the home position, (ii) the switch is in the second position when the collar is in the first activation position, and (iii) the switch is in the third position when the collar is in the second activation position.

5. The electrosurgical instrument of claim 3, wherein the switch comprises a hall effect sensor that is configured to sense when the collar is in the home position, when the collar is in the first activation position, and when the collar is in the second activation position.

6. The electrosurgical instrument of claim 3, wherein the switch comprises a first electrical contact, a second electrical contact, and a third electrical contact,
wherein the collar comprises one or more conductive elements,
wherein, when the collar is in the first activation position, the one or more conductive elements electrically contact the first electrical contact and the second electrical contact, and
wherein, when the collar is in the second activation position, the one or more conductive elements electrically contact the second electrical contact and the third electrical contact.

7. The electrosurgical instrument of claim 6, wherein the switch further comprises a plurality of flaps that cover respective ones of the first electrical contact and the third electrical contact, and
wherein the collar is configured to move the plurality of flaps to expose the respective ones of the first electrical contact and the third electrical contact responsive to the collar moving relative to the housing.

8. The electrosurgical instrument of any one of claims 1-7, wherein the housing comprises a recessed portion,
wherein the recessed portion has cross-sectional dimensions that are less than cross-sectional dimensions of the housing on opposing sides of the recessed portion, and
wherein the collar is disposed and movable in the recessed portion of the housing.

9. The electrosurgical instrument of any one of claims 1-8, wherein a central portion of the collar has a concave shape.

10. The electrosurgical instrument of any one of claims 1-8, wherein a central portion of the collar has a convex shape.

11. The electrosurgical instrument of any one of claims 1-10, wherein the collar comprises a grip portion, and
wherein the grip portion has a coefficient of friction that is greater than a coefficient of friction of at least a portion of the housing.

12. The electrosurgical instrument of any one of claims 1-11, wherein the housing comprises a grip section located proximal on an exterior surface of the housing and proximal of the collar,
wherein the grip section has a coefficient of friction that is greater than a coefficient of friction of at least a portion of the housing.

13. The electrosurgical instrument of any one of claims 1-12, wherein the user activation device further comprises a biasing member that biases the collar towards the home position.

14. The electrosurgical instrument of any one of claims 1-13, wherein the collar comprises a plurality of segments that are independently movable relative to each other and the housing.

15. The electrosurgical instrument of claim 14, wherein the switch comprises a plurality of switches, wherein each segment is configured to actuate a respective one of the plurality of switches.

16. A method of operating an electrosurgical instrument, comprising:
coupling a power cord of an electrosurgical instrument to an electrosurgical generator, wherein the electrosurgical instrument comprises:
a housing extending from a proximal end to a distal end,
an electrosurgical electrode extending from the distal end of the housing, and
a user activation device disposed between the proximal end of the housing and the distal end of the housing, wherein the user activation device is operable to control a supply of electrosurgical energy to the electrosurgical electrode, wherein the user activation device comprises a collar that extends around at least a half of a circumference of the housing, wherein the collar is movable between a home position relative to the housing and a first activation position relative to the housing, wherein the user activation device further comprises a switch that is configured to control the supply of the electrosurgical energy to the electrosurgical electrode responsive to the collar moving from the home position to the first activation position;
moving the collar from the home position to the first activation position; and
responsive to moving the collar from the home position to the first activation position, supplying the electrosurgical energy from the electrosurgical generator to the electrosurgical electrode.

17. The method of claim 16, further comprising:
after supplying the electrosurgical energy, moving the collar from the first activation position to the home position; and
responsive to moving the collar from the first activation position to the home position, ceasing the supply of the electrosurgical energy to the electrosurgical electrode.

18. The method of claim 17, wherein moving the collar from the first activation position to the home position comprises using a biasing member to automatically move the collar from the first activation position to the home position when a user releases the collar.

19. The method of any one of claims 16-18, wherein supplying the electrosurgical energy from the electrosurgical generator to the electrosurgical electrode responsive to moving the collar from the home position to the first activation position comprises supplying the electrosurgical energy with a first waveform.

20. The method of claim 19, further comprising moving the collar from the home position to a second activation position, wherein the home position is between the first activation position and the second activation position, and
responsive to moving the collar from the home position to the second activation position, supplying the electrosurgical energy to the electrosurgical electrode with a second waveform,
wherein the first waveform is different from the second waveform.

21. The method of any one of claim 16-20, wherein the switch comprises a rocker switch, and
wherein moving the collar from the home position to the first activation position comprises mechanically engaging a rocker paddle of the rocker switch to depress a first contact of the rocker switch.

22. The method of any one of claim 16-20, wherein moving the collar from the home position to the first activation position comprises closing a circuit between a first electrical contact of the switch, a second electrical contact of the switch, and a conductive element of the collar.

23. The method of any one of claims 16-20, further comprising:
sensing, by a Hall Effect sensor, that the collar is in the home position;
responsive to sensing that the collar is in the home position, preventing supply of the electrosurgical energy to the electrosurgical electrode; and
sensing, by the Hall Effect sensor, that the collar is in the first activation position,
wherein supplying the electrosurgical energy from the electrosurgical generator to the electrosurgical electrode is responsive to sensing that the collar is in the first activation position.

24. The method of any one of claims 16-23, wherein the collar comprises a plurality of segments that are independently movable relative to each other and the housing, and
wherein moving the collar from the home position to the first activation position comprises moving one segment of the plurality of segments relative to another segment of the plurality of segments.

25. A method of making an electrosurgical instrument, comprising:
forming a housing extending from a proximal end to a distal end;
coupling an electrosurgical electrode to the housing such that the electrosurgical electrode extends from the distal end of the housing; and
coupling a user activation device to the housing between the proximal end of the housing and the distal end of the housing, wherein the user activation device is operable to control a supply of electrosurgical energy to the electrosurgical electrode,
wherein the user activation device comprises:
a collar that extends around at least a half of a circumference of the housing, wherein the collar is movable between a home position relative to the housing and a first activation position relative to the housing, and
a switch that is configured to control the supply of the electrosurgical energy to the electrosurgical electrode responsive to the collar moving from the home position to the first activation position.

26. An electrosurgical device, comprising:
a housing extending between a proximal end and a distal end;
an electrosurgical electrode extending from the distal end of the housing; and
a first user activation device that is operable to control a supply of electrosurgical energy to the electrosurgical electrode,
wherein the first user activation device extends around at least half of a circumference of the housing.

27. The electrosurgical device of claim 26, wherein the first user activation device extends around an entirety of the circumference of the housing.

28. The electrosurgical device of any one of claims 26-27, wherein the first user activation device comprises a plurality of segments, and
wherein each segment extends around a respective portion of the circumference of the housing.

29. The electrosurgical device of claim 28, wherein only one segment of the plurality of segments is operable at a time.

30. The electrosurgical device of claim 29, wherein the first user activation device is configured such that, when one segment of the plurality of segments is depressed towards the housing, the one segment engages adjacent segments of the plurality of segments and stops the adjacent segments from being depressed towards the housing.

31. The electrosurgical device of any one of claims 28-30, wherein each segment comprises an inner surface that faces the housing, an outer surface that faces away from the housing an opposes the inner surface, a distal surface, a proximal surface, a first lateral surface, and a second lateral surface, and
wherein the inner surface of each segment has a common radius of curvature.

32. The electrosurgical device of any one of claims 3-6, wherein the plurality of segments have the same shape and the same size as each other.

33. The electrosurgical device of any one of claims 28-32, further comprising a plurality of switches, wherein the first user activation device engages the plurality of switches, and each switch is disposed between a respective one of the plurality of segments and the housing and is configured to be actuated to control the supply of electrosurgical energy to the electrosurgical electrode.

34. The electrosurgical device of any one of claim 33, further comprising one or more flexible printed circuit boards, wherein the plurality of switches are configured to cause the one or more flexible printed circuit boards to control the supply of electrosurgical energy to the electrosurgical electrode.

35. The electrosurgical device of any one of claims 28-34, wherein the first user activation device comprises at least one sensor that is configured to sense an extent to which each segment is depressed towards the housing.

36. The electrosurgical device of claim 35, wherein the at least one sensor comprises at least one device selected from a group consisting of: one or more pressure sensors, one or more optical sensors, one or more capacitive sensors, one or more inductive sensors, one or more infrared sensors, one or more piezoelectric sensors, one or more ultrasonic sensors, one or more resistive sensors, and one or more image sensors.

37. The electrosurgical device of any one of claims 26-36, further comprising a second user activation device that is offset along a longitudinal axis of the housing relative to the first user activation device,
wherein the longitudinal axis of the housing extends between the proximal end and the distal end of the housing, and
wherein the second user activation device extends around at least half of the circumference of the housing.

38. The electrosurgical device of claim 37, wherein the first user activation device is operable to supply the electrosurgical energy with a first waveform that is configured for cutting tissue,
wherein the second user activation device is operable to supply the electrosurgical energy with a second waveform that is configured for coagulating tissue, and
wherein the first waveform is different than the second waveform.

39. The electrosurgical device of claim 38, wherein the first user activation device comprises at least one first sensor that is configured to sense a first distance by which the first user activation device is depressed towards the housing, and
wherein the second user activation device comprises at least one second sensor that is configured to sense a second distance by which the second user activation device is depressed towards the housing.

40. The electrosurgical device of claim 39, further comprising a controller in communication with the first sensor and the second sensor,
wherein the controller is configured to:
receive, from the at least one first sensor, a first signal indicative of the first distance sensed by the first sensor at a first time,
receive, from the at least one second sensor, a second signal indicative of the second distance sensed by the second sensor at the first time,
make a determination, based on the first signal and the second signal, which of the first distance and the second distance is greater,
if the determination is that the first distance is greater than the second distance, then supply the electrosurgical energy with the first waveform, and
if the determination is that the second distance is greater than the first distance, then supply the electrosurgical energy with the second waveform.

41. The electrosurgical device of any one of claims 26-40, wherein the first user activation device is disposed in a channel in the housing, and
wherein the channel is recessed relative to an exterior surface of the housing.

42. A method for operating an electrosurgical device, comprising:
providing an electrosurgical device, the electrosurgical device comprising:
a housing extending between a proximal end and a distal end;
an electrosurgical electrode extending from the distal end of the housing; and
a first user activation device extending around at least half of a circumference of the housing;
positioning and orienting the electrosurgical electrode at a surgical site; and
operating the first user activation device to control a supply of electrosurgical energy to the electrosurgical electrode.

43. The method of claim 42, wherein the first user activation device extends around an entirety of the circumference of the housing.

44. The method of any one of claims 42-43, wherein the first user activation device comprises a plurality of segments,
wherein each segment extends around a respective portion of the circumference of the housing, and
operating the first user activation device comprises operating one of the segments.

45. The method of claim 44, wherein only one segment of the plurality of segments is operable at a time.

46. The method of claim 45, wherein the first user activation device is configured such that, when one segment of the plurality of segments is depressed towards the housing, the one segment engages adjacent segments of the plurality of segments and stops the adjacent segments from being depressed towards the housing.

47. The method of any one of claims 44-46, wherein each segment comprises an inner surface that faces the housing, an outer surface that faces away from the housing an opposes the inner surface, a distal surface, a proximal surface, a first lateral surface, and a second lateral surface, and
wherein the inner surface of each segment has a common radius of curvature.

48. The method of any one of claims 44-47, wherein the plurality of segments have the same shape and the same size as each other.

49. The method of any one of claims 44-48, wherein the electrosurgical device further comprises a plurality of switches, the first user activation device engages the plurality of switches, and each switch is disposed between a respective one of the plurality of segments and the housing and is configured to be actuated to control the supply of electrosurgical energy to the electrosurgical electrode.

50. The method of any one of claim 49, wherein the electrosurgical device further comprises one or more flexible printed circuit boards, wherein the plurality of switches are configured to cause the one or more flexible printed circuit boards to control the supply of electrosurgical energy to the electrosurgical electrode.

51. The method of any one of claims 44-50, wherein the first user activation device comprises at least one sensor that is configured to sense an extent to which each segment is depressed towards the housing.

52. The method of claim 51, wherein the at least one sensor comprises at least one device selected from a group consisting of: one or more pressure sensors, one or more optical sensors, one or more capacitive sensors, one or more inductive sensors, one or more infrared sensors, one or more piezoelectric sensors, one or more ultrasonic sensors, one or more resistive sensors, and one or more image sensors.

53. The method of any one of claims 42-52, further comprising a second user activation device that is offset along a longitudinal axis of the housing relative to the first user activation device,
wherein the longitudinal axis of the housing extends between the proximal end and the distal end of the housing, and
wherein the second user activation device extends around at least half of the circumference of the housing.

54. The method of claim 53, wherein operating the first user activation device supplies the electrosurgical energy with a first waveform that is configured for cutting tissue,
wherein the second user activation device is operable to supply the electrosurgical energy with a second waveform that is configured for coagulating tissue, and
wherein the first waveform is different than the second waveform.

55. The method of claim 54, wherein the first user activation device comprises at least one first sensor that is configured to sense a first distance by which the first user activation device is depressed towards the housing, and
wherein the second user activation device comprises at least one second sensor that is configured to sense a second distance by which the second user activation device is depressed towards the housing.

56. The method of claim 55, wherein the electrosurgical device includes a controller in communication with the first sensor and the second sensor, and
wherein the controller is configured to:
receive, from the at least one first sensor, a first signal indicative of the first distance sensed by the first sensor at a first time,
receive, from the at least one second sensor, a second signal indicative of the second distance sensed by the second sensor at the first time,
make a determination, based on the first signal and the second signal, which of the first distance and the second distance is greater,
if the determination is that the first distance is greater than the second distance, then supply the electrosurgical energy with the first waveform, and
if the determination is that the second distance is greater than the first distance, then supply the electrosurgical energy with the second waveform.

57. The method of any one of claims 42-56, wherein the first user activation device is disposed in a channel in the housing, and
wherein the channel is recessed relative to an exterior surface of the housing.

58. A method for assembling an electrosurgical device, comprising:
providing a housing extending between a proximal end and a distal end;
coupling an electrosurgical electrode to the housing such that the electrosurgical electrode extends from the distal end of the housing; and
coupling a first user activation device to the housing wherein the first user activation device extends around at least half of a circumference of the housing and is operable to control a supply of electrosurgical energy to the electrosurgical electrode.

59. The method of claim 58, wherein the first user activation device extends around an entirety of the circumference of the housing.

60. The method of any one of claims 58-59, further comprising assembling the first user activation device from a plurality of segments,
wherein each segment extends around a respective portion of the circumference of the housing.

61. The method of claim 60, further comprising configuring the first user activation device such that only one segment of the plurality of segments is operable at a time.

62. The method of claim 61, further comprising configuring the first user activation device such that, when one segment of the plurality of segments is depressed towards the housing, the one segment engages adjacent segments of the plurality of segments and stops the adjacent segments from being depressed towards the housing.

63. The method of any one of claims 60-62, wherein each segment comprises an inner surface that faces the housing, an outer surface that faces away from the housing an opposes the inner surface, a distal surface, a proximal surface, a first lateral surface, and a second lateral surface, and
wherein the inner surface of each segment has a common radius of curvature.

64. The method of any one of claims 60-63, wherein the plurality of segments have the same shape and the same size as each other.

65. The method of any one of claims 60-64, further comprising coupling the first user activation device to a plurality of switches, wherein each switch is disposed between a respective one of the plurality of segments and the housing.

66. The method of claim 65, wherein further comprising coupling the plurality of switches to one or more flexible printed circuit boards, wherein the plurality of switches are configured to cause the one or more flexible printed circuit boards to control the supply of electrosurgical energy to the electrosurgical electrode.

67. The method of any one of claims 60-66, further comprising providing the first user activation device with at least one sensor that is configured to sense an extent to which each segment is depressed towards the housing.

68. The method of claim 67, wherein the at least one sensor comprises at least one device selected from a group consisting of: one or more pressure sensors, one or more optical sensors, one or more capacitive sensors, one or more inductive sensors, one or more infrared sensors, one or more piezoelectric sensors, one or more ultrasonic sensors, one or more resistive sensors, and one or more image sensors.

69. The method of any one of claims 58-68, further comprising coupling a second user activation device to the housing,
wherein the second user activation device is offset along a longitudinal axis of the housing relative to the first user activation device,
wherein the longitudinal axis of the housing extends between the proximal end and the distal end of the housing, and
wherein the second user activation device extends around at least half of the circumference of the housing.

70. The method of claim 69, wherein the first user activation device is operable to supply the electrosurgical energy with a first waveform that is configured for cutting tissue,
wherein the second user activation device is operable to supply the electrosurgical energy with a second waveform that is configured for coagulating tissue, and
wherein the first waveform is different than the second waveform.

71. The method of claim 70, further comprising:
providing the first user activation device with at least one first sensor that is configured to sense a first distance by which the first user activation device is depressed towards the housing; and
providing the second user activation device with at least one second sensor that is configured to sense a second distance by which the second user activation device is depressed towards the housing.

72. The method of claim 71, further comprising providing a controller in communication with the first sensor and the second sensor,
wherein the controller is configured to:
receive, from the at least one first sensor, a first signal indicative of the first distance sensed by the first sensor at a first time,
receive, from the at least one second sensor, a second signal indicative of the second distance sensed by the second sensor at the first time,
make a determination, based on the first signal and the second signal, which of the first distance and the second distance is greater,
if the determination is that the first distance is greater than the second distance, then supply the electrosurgical energy with the first waveform, and
if the determination is that the second distance is greater than the first distance, then supply the electrosurgical energy with the second waveform.

73. The method of any one of claims 58-72, wherein coupling the first user activation device to the housing comprises positioning the first user activation device in a channel in the housing, and
wherein the channel is recessed relative to an exterior surface of the housing.
